(19) United States
(12) Patent Application Publication
(10) Pub. No.: US 2026/0363842 A1
(43) Pub. Date: Nov. 19, 2026

(54) HANDS-FREE ACTIVATION AND DEACTIVATION OF AN IMPLANTABLE MEDICAL DEVICE

(71) Applicant: Boston Scientific Scrimed, Inc., Maple Grove, MN (US)

(72) Inventors: Hernan Altman, Kiryat Tiv'on (IL); Jeffrey Albert Meganck, North Grafton, MA (US); Natalie Ann Borgos, Roseville, MN (US)

(21) Appl. No.: 19/669,385

(22) Filed: May 6, 2026

Related U.S. Application Data
(60) Provisional application No. 63/805,059, filed on May 13, 2025.

(51) Int. Cl. A61F 2/00

(52) U.S. Cl. CPC A61F 2/004; A61F 2250/0085

(57) ABSTRACT

According to an aspect, a device may include an inflatable member. A device may include a fluid reservoir. A device may include an electronic pump device configured to transfer fluid between the inflatable member and the fluid reservoir. A device may include a sensor device configured to be integrated with or coupled to a host device, where the sensor device is configured to receive sensor data, detect a user intent state based on the sensor data, and, in response to the user intent state being detected, generate a control signal. The control signal is configured to cause the electronic pump device to deflate the inflatable member.

### CROSS-REFERENCE TO RELATED APPLICATION

This application claims priority to U.S. Provisional Patent Application No. 63/805,059, filed on May 13, 2025, entitled “HANDS-FREE ACTIVATION AND DEACTIVATION OF AN IMPLANTABLE MEDICAL DEVICE”, the disclosure of which is incorporated by reference herein in its entirety.

### TECHNICAL FIELD

This disclosure relates generally to hands-free activation and deactivation of an implantable medical device.

### BACKGROUND

Some implantable medical devices have a pump device, which, when operated by a user, causes a transfer of fluid between a fluid reservoir and an inflatable member. In some examples, implantable medical devices require users to manually pump fluid into the sphincter or use a smartphone app, which can be difficult for elderly or physically impaired individuals.

### SUMMARY

This disclosure relates to a system that includes a sensor device and an implantable medical device with an electronic pump device, where the sensor device can detect a user intent state (e.g., whether the user intends to control the implantable medical device) based on sensor data. In some examples, the implantable medical device is a urinary control device with an inflatable cuff. The sensor device can generate and send a control signal to the electronic pump device, which causes the electronic pump device to transfer fluid between a fluid reservoir and an inflatable member. The sensor device may be incorporated into or coupled to a host device such as a toilet seat, a toilet bowl, or a urinal pad. According to the techniques discussed herein, the implantable medical device may be controlled without a patient using their hands to operate an external controller such as an application on a smartphone.

In some aspects, the techniques described herein relate to an implantable medical device including: an inflatable member; a fluid reservoir; an electronic pump device configured to transfer fluid between the inflatable member and the fluid reservoir; and a sensor device configured to be integrated with or coupled to a host device, the sensor device configured to: receive sensor data; detect a user intent state based on the sensor data; and in response to the user intent state being detected, generate a control signal, the control signal configured to cause the electronic pump device to deflate the inflatable member.

In some aspects, the techniques described herein relate to a method including: receiving sensor data from a sensor of a sensor device; detecting a user intent state based on the sensor data; in response to the user intent state being detected, generating a control signal; and in response to the control signal, transferring fluid from an inflatable member to a fluid reservoir.

In some aspects, the techniques described herein relate to an implantable medical device including: an electronic pump device configured to transfer fluid between an inflatable member and a fluid reservoir; and a sensor device configured to be integrated with or coupled to a host device, the sensor device configured to generate sensor data, the electronic pump device or the sensor device configured to: detect a user intent state based on the sensor data; in response to the user intent state being detected, generate a control signal; and in response to the control signal, initiate inflation or deflation of the inflatable member by activating one or more pumps of the electronic pump device.

### BRIEF DESCRIPTION OF THE DRAWINGS

FIG. 1A illustrates an implantable medical device with an electrical pump device for transferring fluid between an inflatable member and a fluid reservoir according to an aspect.

FIG. 1B illustrates examples of a sensor according to an aspect.

FIG. 1C illustrates examples of a sensor according to another aspect.

FIG. 2 illustrates an example of a host device integrated with a sensor device configured to communicate with an implantable medical device according to an aspect.

FIG. 3 illustrates an example of another host device according to an aspect.

FIG. 4A an example of a fluidic manifold configured to attach a pump of an implantable medical device according to an aspect.

FIG. 4B illustrates a circuit substrate coupled to the fluidic manifold according to an aspect.

FIG. 4C illustrates a perspective of the fluidic manifold according to an aspect.

FIG. 4D illustrates a perspective of the fluidic manifold according to another aspect.

FIG. 4E illustrates a perspective of a circuit substrate coupled to a fluidic manifold according to an aspect.

FIG. 5 illustrates an implantable medical device according to another aspect.

FIG. 6 illustrates an exploded view of a housing of an electronic pump device according to an aspect.

FIG. 7 illustrates a perspective of an inflatable penile prosthesis according to an aspect.

FIG. 8 illustrates an example of an artificial urinary sphincter device according to an aspect.

FIG. 9 illustrates a flowchart depicting example operations of controlling a fluidic device of an electronic pump device of an implantable medical device according to an aspect.

### DETAILED DESCRIPTION

This disclosure relates to an implantable medical device with an electronic pump device configured to transfer fluid between a fluid reservoir and an inflatable member. The implantable medical device is configured to communicate with a sensor device to enable activation or deactivation of the implantable medical device. In some examples, the activation or deactivation (or a portion thereof) is handsfree (e.g., without the use of the patient’s hands). In some examples, instead of using an external controller (e.g., an application on a user device), the implantable medical device may be activated or deactivated by using sensor data. Activation of the implantable medical device may refer to transferring fluid from the inflatable member to the fluid reservoir (or vice versa). Deactivation of the implantable medical device may refer to transferring fluid from the fluid reservoir to the inflatable member (or vice versa).

The sensor device, or, in some examples, the sensor device in combination with the electronic pump device automatically detects a user intent state, e.g., when a patient is in a position to urinate. In response to the detection of the user intent state, a control signal may be generated to control the implantable medical device, e.g., to transfer fluid between the fluid reservoir and the inflatable member. In some examples, the sensor device is integrated into or coupled to a host device such as a toilet seat, toilet bowl, or urinal pad. The host device may include other devices such as a medical assist device or an override device. In some examples, the host device includes a catheterization aid host device. In some examples, the sensor device detects a user intent state by detecting the presence of the implantable medical device. The detection of the user intent state may be based on sensor data, which may be generated by one or more sensors on the sensor device.

In further detail, the sensor device may include one or more sensors configured to generate sensor data. The type of sensor data may depend upon the type of sensor(s) integrated into the sensor device. The sensor device may include a signal generator. The signal generator detects a user intent state based on the sensor data. The user intent state may indicate that the user intends to operate the implantable medical device. In some examples, in the case of a urinary control device, the user intent state may indicate that the user intends to urinate. In some examples, the signal generator detects a user intent signal based on a sensed signal or a sensed signal achieving a threshold level. The sensed signal may be wireless signal (e.g., a radio frequency signal, a Wi-Fi signal, a Bluetooth signal, an NFC signal, an ultrasonic signal, etc.), a pressure signal, presence of a magnetic field, or a capacitance field. In response to the detection of the user intent state, the sensor device may generate a control signal. The sensor device may transmit the control signal to the electronic pump device. In response to the control signal, the electronic pump device may deflate (or inflate) the inflatable member by activating one or more pumps and/or one or more valves to transfer fluid from the inflatable member to the fluid reservoir (or vice versa). In some examples, the electronic pump device receives the sensor data, and the electronic pump device detects the user intent state based on the sensor data.

The sensor device may include one or more sensors included within or coupled to the host device. In some examples, the host device includes one sensor. In some examples, the host device includes two or more sensors. In some examples, the host device includes two or more sensors of different types, e.g., a pressure sensor and a microphone. In some examples, the sensor device includes a transceiver. In some examples, the transceiver includes a wireless signal detector configured to detect the presence of a wireless signal such as a Wi-Fi signal, a Bluetooth signal, or a near-field communication (NFC) signal. In some examples, the sensor device includes a pressure sensor. In some examples, the sensor device includes a radio-frequency identification (RFID) sensor. In some examples, the sensor device includes a microphone. In some examples, the sensor device includes a capacitive sensor. In some examples, the sensor device includes an inductive sensor. In some examples, the sensor device includes an ultrasonic sensor. In some examples, the sensor device includes a light sensor. In some examples, the sensor device includes a strain gage. In some examples, the sensor device includes an impedance sensor. In some examples, the sensor device includes a chemical sensor.

In some examples, a dedicated toilet seat is installed in the patient’s home, replacing the standard one. In some examples, the sensor device can be coupled to (e.g., clipped onto) the toilet tank (or another part of the toilet). In some examples, the seat includes or is coupled to the sensor device which can detect the implantable medical device when the patient is seated on the toilet and ready for voiding. In some examples, the sensor device may be mounted inside the toilet bowl or inside a urinal. In some examples, the sensor device may be attached to the toilet at a location outside of the bowl (e.g., similar to a bidet control mechanism). In some examples, the sensor device is integrated to or coupled to a urinal pad. In some examples, the sensor device may detect the presence of the implantable medical device using Wi-Fi, RFID, or other passive or active methods. In some examples, the sensor device includes an RFID reader configured to read an RFID tag on the implantable medical device. In some examples, the sensor device includes a light sensor configured to detect changes in illumination. In some examples, voice detection may be added as an activation and/or confirmation method.

In some examples, the sensor device may support automatic de-activation when contact is lost, and/or when the pressure sensor is deactivated (e.g., a pressure reading being less than a threshold level), etc. The sensor device may also include one or more indicators that provide feedback to the patient about connectivity, detected user intent state, and/or inflation mode or deflation mode of the implantable medical device 100. Automatic fluid reverse flow may be also supported, reengaging the artificial sphincter when the patient leaves the seat.

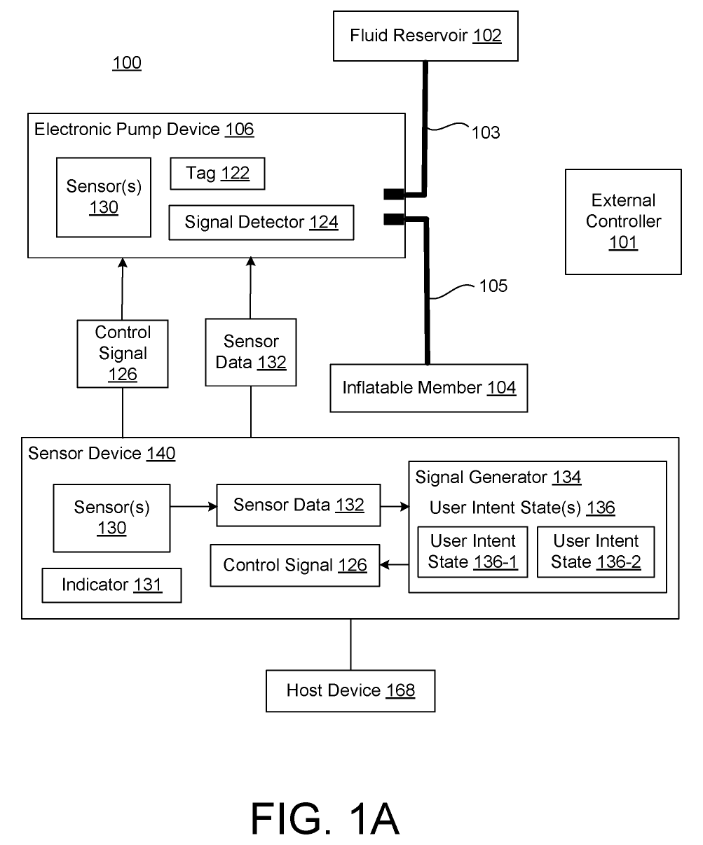

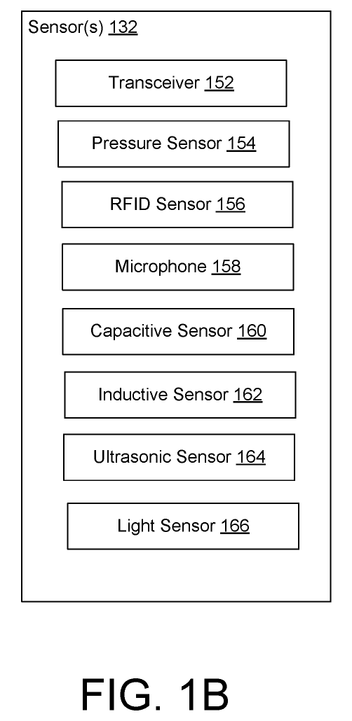

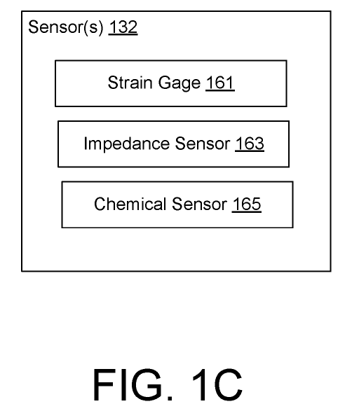

FIGS. 1A-1C illustrate an implantable medical device 100 according to an aspect. The implantable medical device 100 includes a sensor device 140 configured to communicate with an electronic pump device 106 to enable activation or deactivation of the implantable medical device 100. In some examples, the sensor device 140 may reduce or eliminate the need of the user to physically operate a control (e.g., pushing a button, operating a smartphone app or other controller). In some examples, the use of the sensor device 140 may enable handsfree activation or deactivation of the implantable medical device 100. In some examples, some operations or functions of the implantable medical device 100 may be controlled without the use of the patient’s hands while other operations or functions of the implantable medical device 100 may be controlled with the patient’s hands. In some examples, the activation or deactivation of the implantable medical device 100 can be controlled based on sensor data 132. In some examples, the implantable medical device 100 may operate in a handsfree mode in which the activation or deactivation is automatically detected using the sensor data 132. In some examples, automatic activation or deactivation may occur without voice commands. In some examples, activation or deactivation may be based on a voice command. In some examples, a voice command may be used to confirm a user intent state.

In some examples, instead of using an external controller 101 (e.g., an application on a user device), the implantable medical device 100 may be activated or deactivated by using sensor data 132. Activation of the implantable medical device 100 may refer to transferring fluid from an inflatable member 104 to a fluid reservoir 102 (or vice versa). Deactivation of the implantable medical device 100 may refer to transferring fluid from the fluid reservoir 102 to the inflatable member 104 (or vice versa).

In some examples, the implantable medical device 100 includes a urology device. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100 such as respiratory devices, cardiovascular devices, gastrointestinal devices, ophthalmic devices, drug delivery devices, and/or diagnostic devices. In some examples, the implantable medical device 100 may be a stimulation device. In some examples, the implantable medical device 100 may be a neuromodulation device.

The implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen, scrotum, or pelvic cavity of the user (e.g., the fluid reservoir 102 may be implanted in the lower portion of the user’s abdominal cavity or the upper portion of the user’s pelvic cavity). In some examples, at least a portion of the electronic pump device 106 may be implemented in the patient’s body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals (e.g., control signals) from an external controller 101. The external controller 101 may be any type of component that can communicate with the electronic pump device 106. The external controller 101 may be a computer, smartphone, tablet, pendant, key fob, etc. A user may use the external controller 101 to control the implantable medical device 100. In some examples, the user may use the external controller 101 to inflate or deflate the inflatable member 104. However, in some examples, instead of using the external controller 101 to transfer fluid between the fluid reservoir 102 and the inflatable member 104, the electronic pump device 106 communicates with a sensor device 140 to automatically activate or deactivate the implantable medical device 100 without the patient using their hands.

The electronic pump device 106 includes a housing. The housing includes a fluidic manifold that attaches fluid transfer and pressure regulating components. In some examples, the fluidic manifold includes a conductive-based material (e.g., a metal-based material). In some examples, the fluidic manifold is a titanium frame. The fluidic manifold may attach a circuit substrate with electronic components. The fluidic manifold may attach one or more fluidic devices such pumps and/or valves. A pump includes an electronically controlled pump. In some examples, the pump includes a piezoelectric diaphragm pump. A valve may be an electronically controlled valve. A valve may be a piezo-electric diaphragm valve.

Based on sensor data 132 generated by one or more sensors 130 of the sensor device 140, a control signal 126 may be generated. The electronic pump device 106 includes a signal detector 124. The signal detector 124 may receive or detect the control signal 126. In some examples, the signal detector 124 can be referred to as a signal processor. The signal detector 124 may detect the control signal 126 and generate an output signal that causes the electronic pump device 106 to perform an action. The control signal 126 may cause the electronic pump device 106 to transfer fluid from the inflatable member 104 to the fluid reservoir 102 (or vice versa). In some examples, as further explained below, the signal detector 124 may include one or more capabilities of the signal generator 134 (e.g., detecting a user intent state 136 and generating a control signal 126).

For example, the sensor device 140, or, in some examples, the sensor device 140 in combination with the electronic pump device 106 automatically detects a user intent state 136. A user intent state 136 may be an instance of when the user intends to operate the implantable medical device 100. A user intent state 136 may refer to a condition or mode that indicates the user's desire or readiness to initiate a specific action related to the implantable medical device 100. In some examples, a user intent state 136 may refer to a state in which the user intends to transfer fluid to or from the inflatable member 104. In some examples, the user intent state 136 includes a user intent state 136-1 and a user intent state 136-2. The user intent state 136-1 may be referred to as a first user intent state. The user intent state 136-2 may be referred to as a second user intent state. Although two user intent states 136 are depicted in FIG. 1A, the implantable medical device 100 may define any number of user intent states 136 that can be detected through the use of sensor data 132 (e.g., one user intent state or any number of user intent states greater than two). The type of user intent state 136 may depend on the implantation of the implantable medical device 100.

In some examples, the user intent state 136-1 is when the user intends to deflate the inflatable member 104 so that the user can urinate. In some examples, the user intent state 136-2 is when the user intends to inflate the inflatable member 104 when the user is finished urinating. In some examples, the user intent state 136-1 is when the user intent to inflate the inflatable cylinders of the prosthesis. In some examples, the user intent state 136-2 is when the user intent to deflate the inflatable cylinders of the prosthesis. The user intent state 136 may include other states such as a user intent state (e.g., a third user intent state) relating to preparing the implantable medical device for catheter insertion. In some examples, the user intent state 136 include a user intent state (e.g., a fourth user intent state) that allow authorized medical personnel to manually control the implantable medical device, overriding the automatic sensor-based control.

In some examples, the implantable medical device 100 may be configured to minimize or prevent unintended activation or deactivation of the inflatable member 104. For example, the implantable medical device 100 may prevent unintended deflation of the inflatable member 104, which could lead to incontinence if it occurs accidentally, such as when a user merely walks by the sensor device 140 integrated into a host device 168 like a toilet. Therefore, in some examples, the system is configured to confirm the validity of the detected user intent state 136-1 before generating the control signal 126 for deflation. This confirmation can be achieved through various mechanisms. In some examples, a separate user event state or condition may be required to confirm the intent, such as a user performing a specific action (e.g., a sustained touch on a capacitive sensor, a voice command, or a deliberate posture). In other examples, an algorithm may be employed to determine the applicability and validity of the detected user intent state 136-1 based on factors such as the duration for which the sensor data indicative of the state is detected (e.g., a time-based confirmation), or by analyzing patterns across multiple sensor inputs. For example, the system may require a pressure reading above a threshold to be sustained for a predetermined period, potentially combined with data from other sensors like an inductive or capacitive sensor detecting the presence of the user's body or the implantable device.

The second user intent state 136-2 may correspond to an intention to inflate the inflatable member 104. In the urinary control device use case, this state indicates the user has finished urinating and wishes to reinflate the cuff to restore continence. In some examples, the system may aquatically detect this state and trigger reinflation. In some examples, the user intent state 136-2 is detected based on sensor data 132. In some examples, the detection of user intent state 136-2 can be based on changes in the sensor data 132 indicating that the user is no longer in the position or proximity associated with the deflation state. For example, when a patient leaves the bathroom or moves away from the host device 168 (e.g., standing up from a toilet seat), the sensor device 140 may detect a significant change in sensor data. This change could include the loss of a wireless signal connection to the electronic pump device 106, a decrease in pressure below a threshold level detected by a pressure sensor 154 on a toilet seat, a change in the capacitive or inductive field detected by capacitive sensor 160 or inductive sensor 162, or a change in luminance detected by a light sensor 166. A sustained lack of detection of the pump device 106 by the sensor device 140 for a defined period can also trigger automatic reinflation (user intent state 136-2). Furthermore, data from a chemical sensor 165 indicating the cessation of urine flow can serve as a confirmation that voiding is complete, either triggering or confirming the user intent state 136-2 for reinflation. Upon detection of user intent state 136-2, a control signal 126 is generated to cause the electronic pump device 106 to transfer fluid from the fluid reservoir 102 to the inflatable member 104.

The sensor device 140, or a combination of the sensor device 140 and the electronic pump device 106, includes a signal generator 134 (or equivalent processing capability) configured to analyze the sensor data 132 and detect the user intent states 136-1 and 136-2 based on predefined criteria, thresholds, algorithms, and/or or machine learning models as described in further detail herein. The host device 168 provides a stable platform for the sensor device 140 to reliably gather this data in the user's environment.

In response to the detection of the user intent state 136 (e.g., the user intent state 136-1 or the user intent state 136-2), a control signal 126 may be generated to control the implantable medical device 100, e.g., to transfer fluid between the fluid reservoir 102 and the inflatable member 104. In some examples, the sensor device 140 is integrated into or coupled to a host device 168. In some examples, the sensor device 140 is a sub-component of the host device 168. In some examples, the sensor device 140 is one or more components that can be attached to the host device 168. In some examples, the sensor device 140 is a sub-component that is integrated into the host device 168 during manufacture or assembly of the host device 168. In some examples, the host device 168 and the sensor device 140 are referring to the same device (e.g., instances of the term “sensor device” and be replaced with “host device” or vice versa). In some examples, the host device 168 may be any type of component that includes (or is integrated with) the component(s) of the sensor device 140 (or a portion of the sensor device 140). The host device 168 may include one or more sensors 130. In some examples, the host device 168 includes the signal generator 134 (or a portion of the signal generator 134).

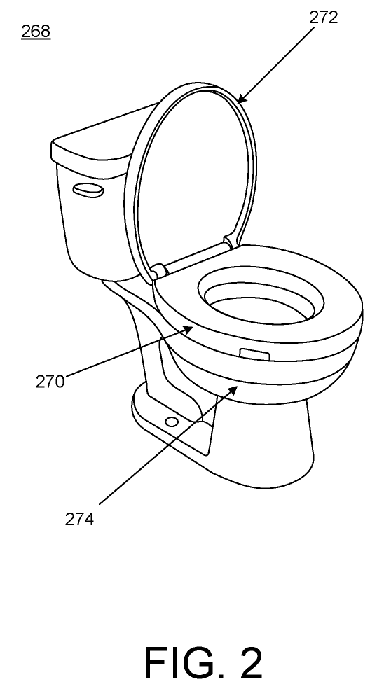

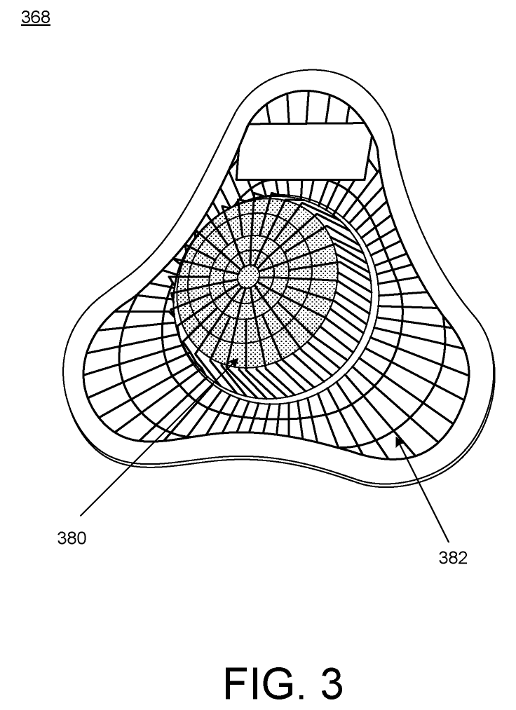

The host device 268 may refer to a structure or object(s) that can attach sensor(s) 130, and, in some examples, a circuit device that includes the signal generator 134. In some examples, the host device 268 may refer to a structure or object(s) within the user's environment that provides a stable and predictable platform or location for the sensor device 140 to reliably gather data related to user intent. In some examples, the host device 268 is a small portable device. In some examples, the host device 268 is a handheld device. In some examples, the host device 268 may be a wearable device or integrated into an article of clothing or jewelry. In some examples, the host device 268 may be a bathroom fixture that the user interacts with during voiding. In some examples, the host device 168 includes a toilet seat (e.g., toilet seat 270 of FIG. 2), a toilet bowl (e.g., toilet bowl 274 of FIG. 2), or a urinal pad (e.g., host device 368 of FIG. 3).

In some examples, the host device 168 includes a specialized medical equipment or an assistive device. In some examples, the host device 168 includes a catheterization aid host device. In some examples, the signal generator 134 detects a user intent state (e.g., a third user intent state) relating to a catheterization mode based on the sensor data 132, and, in response to detection of the third user intent state, the signal generator 134 may generate a control signal 126 to prepare the implantable medical device 100 for catheter insertion. In some examples, the host device 168 includes a manual activation switch that the user presses to confirm their intent to catheterize.

In some examples, the host device 168 includes a medical override device. The medical override device may allow authorized medical personnel to manually control the implantable medical device 100, overriding the automatic sensor-based control. The medical override device may include security features, such as passcode authentication or biometric identification, to prevent unauthorized use. The medical override device may also display device status information (e.g., cuff pressure, battery level) and provide manual controls (e.g., buttons or a touchscreen) for inflation and deflation of the inflatable member 104. In some examples, the medical personnel would interact with the medical override device to trigger a user intent state (e.g., a fourth user intent state) relating to medical override mode and generate a control signal 126 to inflate or deflate the inflatable member 104.

In some examples, the sensor device 140 includes a signal generator 134 configured to detect a user intent state 136 based on the sensor data 132. In some examples, the signal generator 134 is included in the electronic pump device 106, where the electronic pump device 106 receives the sensor data 132 and the electronic pump device 106 is configured to detect a user intent state 136 based on the sensor data 132 and generate a control signal 126 based on the detected user intent state 136. In some examples, the signal generator 134 detects the user intent state 136 by detecting the presence of the electronic pump device 106. The detection of the user intent state 136 may be based on sensor data 132, which may be generated by one or more sensors 130 on the sensor device 140.

The sensor device 140 may include one or more sensors 130 configured to generate sensor data 132. The type of sensor data 132 may depend upon the type of sensor(s) 130 integrated into the sensor device 140. The signal generator 134 detects a user intent state 136 based on the sensor data 132, where the user intent state 136 may indicate that the user intends to operate the implantable medical device 100. In some examples, in the case of a urinary control device, the user intent state 136 may indicate that the user intends to urinate. In some examples, sensor data 132 includes a sensed signal, and the signal generator 134 detects a user intent state 136 based on the sensed signal or the sensed signal achieving a threshold level.

The sensed signal may be wireless signal (e.g., a radio frequency signal, a Wi-Fi signal, a Bluetooth signal, an NFC signal, an ultrasonic signal, etc.), a pressure signal, a presence of a magnetic field, or a capacitance field. In response to the detection of the user intent state 136, the signal generator 134 may generate a control signal 126. In some examples, the sensor device 140 may transmit the control signal 126 to the electronic pump device 106. In response to the control signal 126, the electronic pump device 106 may deflate (or inflate) the inflatable member 104 by activating one or more pumps and/or one or more valves to transfer fluid from the inflatable member 104 to the fluid reservoir 102 (or vice versa). In some examples, the electronic pump device 106 receives the sensor data 132, and the electronic pump device 106 detects the user intent state 136 based on the sensor data 132.

The sensor device 140 may include one or more sensors 130 (or any combination thereof) as shown in FIGS. 1B and 1C. As shown in FIGS. 1B and 1C, the sensor device 140 may include one or more of the following: a transceiver 152, a pressure sensor 154, a RFID sensor 156 (e.g., RFID reader or a RFIF emitter and reader), a microphone 158, a capacitive sensor 160, an inductive sensor 162, an ultrasonic sensor 164, a light sensor 166, a strain gage 161, an impedance sensor 163, and/or a chemical sensor 165. In some examples, the sensor device 140 may include one or more other sensors 130 such as a motion sensor. In some examples, the sensor device 140 includes an accelerometer. In some examples, the sensor device 140 includes a gyroscope. In some examples, the sensor device 140 may include one or more inertial measurement units (IMUs). In some examples, the sensor device 140 includes an electromyography (EMG) sensor. In some examples, the sensor device 140 includes electroencephalography (EEG) Sensor. In some examples, the sensor device 140 includes a near-infrared spectroscopy (NIRS) sensor. In some examples, the sensor device 140 includes an acoustic sensor.

In some examples, the sensor device 140 includes a data fusion engine configured to combine data from multiple sensors 130 and one or more machine-learning (ML) models (e.g., neural-network based models) configured to receive the combined data and detect a user intent state 136-1 or a user intent state 136-2. The data fusion engine is configured to receive and preprocess sensor data 132 from multiple sensors 130, including but not limited to pressure sensor 154, capacitive sensor 160, RFID sensor 156, ultrasonic sensor 164, and/or microphone 158 or any two or more of the sensors 130 described herein. Preprocessing steps may include noise reduction, data cleaning, synchronization, and/or feature extraction. The preprocessed sensor data is then fed into a machine learning (ML) model, such as a recurrent neural network (RNN) or support vector machine (SVM), trained to recognize patterns indicative of different user intent states 136-1 and 136-2. The ML model may output probabilities or confidence scores for each state, and the data fusion engine uses these scores to determine the most likely user intent.

In some examples, as shown in FIG. 1B, the sensor device 140 includes a transceiver 152. In some examples, the transceiver 152 includes a Wi-Fi transceiver. In some examples, the transceiver 152 includes a short-range transceiver such as a Bluetooth transceiver or an NFC transceiver. The transceiver 152 may detect the presence of a wireless signal such as a Wi-Fi signal, a Bluetooth signal, or a near-field communication (NFC) signal. In some examples, the signal generator 134 detects the user intent state 136-1 when the sensor data 132 indicates the presence of a wireless signal. In some examples, the signal generator 134 detects the user intent state 136-2 when the sensor data 132 indicates the lack of a wireless signal or a wireless signal having a strength that is less than a threshold level. In some examples, the signal generator 134 detects a user intent state 136-2 when the user intent state 136-1 has already been detected and there is a change to the sensor data 132 (e.g., a lack of the wireless signal previously detected or having a strength that dropped below a threshold level).

The transceiver 152 is configured to detect one or more types of wireless signals, including but not limited to radiofrequency signals, Wi-Fi signals, Bluetooth signals, Bluetooth Low Energy (BLE) signals, Zigbee signals, near-field communication (NFC) signals, and ultra-wideband (UWB) signals. The transceiver may be capable of detecting the presence of a wireless signal by measuring signal strength (e.g., RSSI), signal patterns, or specific data encoded within the wireless signal. In some examples, the wireless signal is emitted by the electronic pump device 106.

The presence of a wireless signal, particularly from the electronic pump device 106, can indicate a user intent state 136. For example, a signal from the electronic pump device 106 indicates the user is in close proximity to the sensor device 140 (e.g., sitting on a toilet seat), suggesting readiness to use the implantable medical device 100. In some examples, the wireless signal may contain an identifier that specifically confirms the user's identity and/or authorization to use the device.

Conversely, the absence or lack of the wireless signal, or a significant reduction in its signal strength below a predetermined threshold, can be interpreted as a second user intent state (e.g., user intent state 136-2), indicating a desire to initiate a second, opposing action, such as inflating the inflatable member 104. The cessation of the signal transmission (e.g., due to the user moving out of range) may be used as a cue to trigger a change in the device's state. In some examples, the implantable medical device 100 uses a timeout period to enhance reliability and prevent untended actions. For example, the detection of the user intent state 136-2 may occur if the wireless signal remains absent or below the threshold for a specified duration. This may prevent momentary signal drops from causing spurious device activation.

In some examples, the user approaches and sits down, triggering the deflation of the inflatable member 104 (e.g., user intent state 136-1, signal present). After voiding, the user rises or moves away, and the wireless signal is lost. This loss of signal may indicate the user's intent to have the cuff re-inflated to restore continence (e.g., user intent state 136-2, signal absent). Therefore, detecting the absence of the wireless signal provides an automated and hands-free way to manage the cuff state in a manner that aligns with the user's natural behavior and needs

In some examples, the sensor device 140 includes a pressure sensor 154. In some examples, the pressure sensor 154 is configured to generate a pressure reading about a pressure that is applied to the host device 168 (e.g., a toilet seat). In some examples, the signal generator 134 may detect a user intent state 136-1 when the pressure reading is equal to or greater than a threshold level. In some examples, the signal generator 134 may detect a user intent state 136-2 when the pressure reading is less than the threshold level.

For example, the sensor device 140 may include one or more pressure sensors 154 to detect the presence of a user on the host device 168. In some examples, a pressure sensor 154 can detect the pressure applied to a host device 168. In examples where the host device 168 is a toilet seat, the pressure sensor(s) 154 may include piezoresistive sensors or capacitive sensors, which may provide relatively accurate and repeatable measurements under typical bathroom conditions. The pressure sensor 154 is positioned within the toilet seat such that it measures the force exerted on the seating surface. In some examples, multiple pressure sensors are distributed across the seat to provide a more uniform measurement of the user's weight.

When a user sits on the toilet seat, the pressure sensor 154 generates a pressure reading, typically an electrical signal proportional to the applied force. The signal generator 134 receives this pressure reading and may compare the pressure value from the pressure reading to a pressure threshold level (e.g., whether the pressure value is equal to or greater than the threshold level). This threshold level may be determined during a calibration process, considering factors such as the weight distribution of a typical user and the physical characteristics of the toilet seat. For example, the threshold level may be set to a value corresponding to a threshold value to differentiate between the presence of a user and lighter objects.

To improve the robustness of the user intent detection, the signal generator 134 may employ a filtering technique, such as calculating a moving average of the pressure readings over a short time window (e.g., 1-2 seconds). This may help to smooth out any transient pressure fluctuations. Furthermore, the rate of change of the pressure reading can be analyzed to distinguish between a user sitting down and a static load. A rapid increase in pressure suggests a user is sitting, while a slow change or constant pressure may indicate an inanimate object. In some examples, a rapid increase in pressure, which then remains at the higher pressure level for a period of time may indicate a user instant state 136-1 (e.g., to distinguish from an object falling and colliding with the toilet seat. In some examples, the pressure sensor data is combined with data from other sensors. For instance, the system may activate the implantable medical device 100 if the pressure reading exceeds the threshold and an RFID tag is detected, ensuring that only an authorized user can trigger the device.

In some examples, the system may also include one or more fault detection mechanisms. If the pressure sensor 154 fails to provide a valid reading, an error signal may be generated, and the system may revert to a backup control method or alert the user. Examples of typical pressure ranges for a user sitting on a toilet seat might be 400 N to 1200 N, with a threshold set at 300 N. The system might also monitor the rate of pressure change, requiring a change of at least 100 N/second to register as a sitting down event.

In some examples, the sensor device 140 includes a radio-frequency identification (RFID) sensor 156 configure to emit radio waves. In some examples, the electronic pump device 106 includes a tag 122. In response to the detection of the radio wave by the tag 122, the tag 122 may transmit RFID data to the RFID sensor 156, which may include an identifier of the tag 122. In response to the detection of the RFID data, the signal generator 134 may detect a user intent state 136-1. In response to the lack of detection of the RFID data, the signal generator 134 may generate a user intent state 136-2.

In some examples, the sensor device 140 includes an RFID sensor 156 to identify the implantable medical device and infer the presence of an authorized user. The system may employ ultra-high frequency (UHF) passive tags, which offer a good balance of reading range and power efficiency. In some examples, the RFID tag 122 is embedded within the housing of the electronic pump device 106, ensuring it remains with the device. The RFID sensor 156 may be integrated into the host device 168, positioned to increase the likelihood of reading the tag when the user is in the intended position for device use (e.g., on a toilet seat).

In some examples, the RFID tag 122 stores a unique identifier that is transmitted to the RFID sensor 156 when the tag enters the reader's electromagnetic field. This identifier allows the system to verify that the correct implantable device is present. In some examples, the RFID tag 122 may also store encrypted data related to user authorization or device settings, enhancing security and personalization. To minimize power consumption, the RFID sensor 156 may operate in an intermittent mode, periodically emitting radio waves. The system may also incorporate a challenge-response protocol, where the RFID sensor 156 sends a challenge to the RFID tag 122, and the tag 122 must provide a correct response to be authenticated. This may prevent unauthorized activation by simple RFID cloning.

If the RFID sensor 156 fails to detect the RFID tag 122 after a predetermined number of attempts, the system may generate an error signal or prompt the user to reposition themselves. The RFID data can also be combined with data from other sensors. For example, the system may only activate the device if both the RFID tag 122 is detected and the pressure sensor 154 indicates the presence of a user on the host device 168, providing a higher level of confidence in the user's intent.

In some examples, the sensor device 140 includes a microphone 158. The microphone 158 may capture audio data, including audio commands by the patient. In some examples, a patient may provide an audio command (“activate), which, when received, causes the signal generator 134 to detect a user intent state 136-1. In some examples, a patient may provide an audio command (“deactivate”), which, when received, causes the signal generator 134 to detect a user intent state 136-2. In some examples, the sensor device 140 includes a signal processor to process the voice commands detected by the microphone 158. In some examples, the sensor device 140 includes a speaker to provide auditory feedback or confirm a selection.

In some examples, the sensor device 140 incorporates a microphone 158 to enable voice-activated control of the implantable medical device 100. In some examples, the microphone 158 is a noise-canceling microphone positioned to capture the user's voice clearly while minimizing background interference. In some examples, the audio data captured by the microphone 158 is processed by a speech recognition module within the signal generator 134. This module may employ a neural network-based algorithm trained to recognize a predefined set of voice commands. In some embodiments, the speech recognition is performed locally on the sensor device 140 to ensure rapid response times. For enhanced security, the system may include voiceprint analysis, where the user's voice is analyzed to verify their identity before a command is executed. To improve accuracy in noisy environments, the system may use digital noise reduction techniques to filter out background sounds. When a valid voice command is recognized, the system provides auditory feedback to the user, such as a confirmation tone. If a command is not recognized, the system prompts the user to repeat the command.

In some examples, the speech recognition module supports multiple languages, allowing for use by a diverse user population. In some examples, the microphone 158 is activated intermittently to conserve power, triggered by other sensor inputs or by a user-initiated wake-up command. The power consumption of the speech recognition module is optimized through efficient algorithms and hardware implementation.

In some examples, the sensor device 140 includes a capacitive sensor 160. In some examples, the capacitive sensor 160 may generate capacitance data about a capacitance field around or proximate to the sensor device 140. Based on the capacitance data, the signal generator 134 may detect a user intent state 136-1 or a user intent state 136-2 by detecting a change to the capacitance field.

For example, the sensor device 140 may include one or more capacitive sensors 160 to detect the presence and proximity of the user. In some examples, projected capacitive sensing is employed, allowing the capacitive sensor 160 to detect changes through a non-conductive surface of the host device 168. The capacitive sensor 160 may include an array of conductive electrodes arranged to create a capacitance field extending outwards from the host device 168.

When no user is present, the capacitive sensor 160 measures a baseline capacitance. The system monitors (e.g., continuously or periodically monitors) the capacitance data and employs a change detection algorithm to identify significant deviations from this baseline. This algorithm may utilize a threshold-based approach, where a change exceeding a predetermined value is interpreted as a user interaction. To minimize false detections, the system may also analyze the rate of change of capacitance, requiring a rapid change to register as a valid user presence. The sensitivity of the capacitive sensor 160 is configured to detect changes as small as 0.1 picofarads, providing a sensing range of up to 30 cm.

In a toilet seat embodiment, the capacitive sensor 160 is embedded within the seat material. As a user approaches the toilet, the capacitance field begins to change, indicating an impending interaction. When the user sits down, the capacitance field changes significantly, allowing the signal generator 134 to detect user intent state 136-1. As the user rises and moves away, the capacitance field returns to the baseline, indicating user intent state 136-2. In some examples, the capacitive sensor data is fused with data from a pressure sensor 154. The system requires both a change in capacitance and a pressure reading above a threshold to confirm user presence, reducing the likelihood of false triggers.

In some examples, the sensor device 140 includes an inductive sensor 162. The inductive sensor 162 may generate inductive data about a magnetic field around or proximate to the sensor device 140. Based on the inductive data, the signal generator 134 may detect a user intent state 136-1 or a user intent state 136-2 by detecting a change to the magnetic field.

In some examples, the sensor device 140 includes one or more inductive sensors 162 to detect the presence or proximity of a user. In some examples, eddy current sensing is employed, enabling the sensor to detect conductive materials. In some examples, the inductive sensor 162 includes a coil designed to generate a magnetic field.

When no user is present, the inductive sensor 162 establishes a baseline magnetic field. The system monitors (e.g., continuously or periodically monitors) the inductive data, and a change detection algorithm identifies significant deviations from this baseline. This algorithm may use a threshold-based approach, where a change exceeding a predetermined value triggers an event. To minimize false detections, the system may also analyze the rate of change of inductance. The sensitivity of the inductive sensor 162 is configured to detect changes in inductance as small as one microhenry, providing a sensing range of up to ten cm for metallic objects.

In a toilet seat embodiment, the inductive sensor 162 is integrated into the seat. If a user with metallic objects (e.g., belt buckle, metallic implant) approaches or sits on the toilet, the magnetic field changes, allowing the signal generator 134 to detect user intent state 136-1. When the user leaves, the field returns to baseline, indicating user intent state 136-2. In some examples, the inductive sensor data is combined with data from a pressure sensor 154. The system requires both a change in the magnetic field and a pressure reading above a threshold to confirm user presence.

In some examples, the electronic pump device 106 includes a magnet (or a magnetically permeable material) instead of an RFID tag. The magnet may be a small, high-strength neodymium magnet encapsulated in a biocompatible material, such as titanium, and securely housed within the device's housing. The inductive sensor 162 on the sensor device 140 is configured to detect changes in the magnetic field caused by the proximity of this magnet.

When the electronic pump device 106 is brought into close proximity to the sensor device 140 (e.g., when the user is positioned on a toilet seat containing the sensor device), the magnet alters the magnetic field in the vicinity of the inductive sensor 162. The inductive sensor 162 detects this change, and the signal generator 134 interprets it as a first user intent state (e.g., user intent state 136-1), indicating the user's intention to activate the device. Conversely, when the electronic pump device 106 is moved away from the sensor device 140, the magnetic field returns to its baseline level. The inductive sensor 162 detects this change (or lack of change), and the signal generator 134 interprets it as a second user intent state (e.g., user intent state 136-2), indicating the user's intention to deactivate the device or the user's absence. In some examples, the magnet-based approach offers advantages in terms of simplicity and power consumption compared to RFID systems but may have a shorter detection range and require careful shielding to avoid external magnetic interference.

In some examples, the sensor device 140 includes an ultrasonic sensor 164. In some examples, the ultrasonic sensor 164 may detect the presence and proximity of a user or an ultrasonic signal generated by the electronic pump device 106. In some examples, the ultrasonic sensor 164 operates by emitting pulses of ultrasonic sound waves and measuring the time-of-flight of the reflected echoes. An enclosed-type ultrasonic sensor may be used to minimize the influence of environmental factors.

The ultrasonic sensor 164 may be positioned on the host device 168 to provide sufficient coverage of the intended interaction zone. For example, on a toilet seat, the ultrasonic sensor 164 may be mounted at the rear to detect when a user approaches and sits down. The sensor takes measurements periodically at a sampling rate. The system may employ signal processing techniques, including echo filtering and temperature compensation, to ensure accurate distance measurements. A change detection algorithm analyzes the distance data to identify user intent states 136. This algorithm may use a combination of threshold-based detection and rate of change analysis. For instance, a rapid decrease in distance below a threshold indicates a user approaching, while a slow increase indicates the user moving away.

In a toilet seat embodiment, as a user approaches the toilet, the measured distance decreases. When the user sits down, the distance stabilizes, allowing the signal generator 134 to detect user intent state 136-1. As the user rises and moves away, the distance increases, indicating user intent state 136-2. In some examples, the ultrasonic sensor data is combined with data from a pressure sensor 154. The system requires both a change in distance and a pressure reading above a threshold to confirm user presence and initiate device activation.

In some examples, a user intent state 136 is detected using an ultrasonic link between the electronic pump device 106 and the sensor device 140. In some examples, the electronic pump device 106 may include an ultrasonic transmitter, a small transducer that emits ultrasonic waves at a frequency. In some examples, the transmitter is designed for low power consumption, using pulsed operation to minimize energy use. The ultrasonic transmitter may be positioned on the housing of the electronic pump device 106 to increase the likelihood of the signal reaching the sensor device 140 when the user is in proximity to the host device 168. The sensor device 140 includes a corresponding ultrasonic receiver (e.g., ultrasonic sensor 164), tuned to detect the ultrasonic signal. The receiver may be sensitive to ensure reliable detection even at a distance.

When the electronic pump device 106 is within the effective range, the ultrasonic sensor 164 detects the ultrasonic signal. The signal generator 134 interprets the reception of this signal as a first user intent state (e.g., user intent state 136-1), indicating the user's intention to use the device. If the electronic pump device 106 moves out of range, or if the ultrasonic signal is blocked by an obstruction, the ultrasonic sensor 164 no longer detects the ultrasonic signal. The signal generator 134 interprets the absence of the ultrasonic signal as a second user intent state (e.g., a user intent state 136-2), indicating that the user has moved away or does not intend to use the device.

In some examples, the sensor device 140 includes a light sensor 166. The light sensor 166 may generate luminance data about a luminance around or proximate to the sensor device 140. In some examples, the signal generator 134 may detect a user intent state 136-1 using the luminance data by detecting a change in the luminance and/or may detect a user intent state 136-2 by detecting a change in the luminance.

In some examples, the sensor device 140 includes a light sensor 166 to detect changes in the ambient light and infer user presence or actions. In some embodiments, a photodiode is used for its fast response time and sensitivity to a broad spectrum of light. The light sensor 166 is positioned on the host device 168 to capture changes in luminance caused by user movement or actions. The system establishes a baseline luminance reading and monitors for deviations (e.g., significant deviations). The signal generator 134 employs a change detection algorithm that analyzes the magnitude and/or rate of change of the luminance data. For example, a decrease (e.g., a rapid decrease) in luminance below a threshold may indicate a shadow being cast on the sensor, while a gradual increase may indicate a light being turned on. In some examples, the light sensor 166 has a sensitivity in the range of 0.1 to 0.6 lux and a dynamic range of 0.1 to 10,000 lux.

In a toilet seat embodiment, the light sensor 166 is positioned to detect shadows. As a user approaches or sits down, their body casts a shadow, causing a decrease in luminance. The signal generator 134 detects this change and infers user intent state 136-1. When the user moves away, the shadow disappears, and the luminance returns to baseline, indicating user intent state 136-2. In some embodiments, the light sensor data is combined with data from a pressure sensor 154. The system may require a change in luminance and a pressure reading above a threshold to confirm user presence and initiate device activation.

In some examples, while a user approaching or sitting down will cast a shadow causing a decrease in luminance, the toilet seat lid 272 when closed can also cast a shadow on the sensor's location, potentially leading to a false detection of user intent state 136-1 (intent to deflate). To address this, the implementation of the light sensor 166 and the signal generator 134 is configured to provide directionality in the shadow detection. In some examples, the light sensor 166 can be positioned on the toilet seat 270 in a location where it is significantly shadowed by a seated user but minimally affected by the lid 272 in its typical closed position. For example, placing the sensor on the front or side portion of the seat, or orienting its sensing element to have a field of view that is primarily directed upwards or slightly outwards, can help to isolate shadows cast from above (by the user) from those cast from the rear (by the closing lid). In some examples, multiple light sensors 166 can be distributed across the surface of the toilet seat 270. The signal generator 134 can then analyze the pattern of luminance changes detected by this array of sensors. A user sitting down will cast a shadow pattern across the sensors that is likely different in shape, intensity, and timing compared to the shadow pattern caused by the lid closing. The system can be trained or programmed to recognize the characteristic shadow pattern associated with a user's presence as indicative of user intent state 136-1, while ignoring or differently interpreting patterns caused by the lid.

In some examples, the signal generator 134 can analyze not just the magnitude of the luminance decrease but also the rate of change and the duration of the shadow. A user sitting down typically causes a rapid decrease in luminance that stabilizes once they are seated. The lid closing might cause a different speed or profile of luminance change. Furthermore, combining the light sensor data with a pressure sensor 154, as previously described, may provide context. A detected shadow is only interpreted as user intent state 136-1 if it occurs in conjunction with a pressure reading on the seat 270 exceeding the threshold level, confirming that a user is physically present on the seat. If a shadow is detected but no significant pressure is applied, the system can infer that the shadow is likely from the lid or another source and disregard it as an indicator of user intent to operate the medical device.

In some examples, the sensor device 140 incorporates one or more strain gages 161 to detect user interaction with the host device 168 by measuring the deformation caused by the user's presence and movement. The strain gage(s) 161 may be coupled to the underside of the toilet seat at strategic locations to capture the seat's flexure. When a user sits on the toilet seat 270, the strain gage(s) 161 detect the resulting bending, producing a change in resistance proportional to the applied force. The signal generator 134 may monitor the output of the strain gages 161. A relatively rapid increase in strain, exceeding a predefined threshold, may be interpreted as the user sitting down. This action is correlated with the user intent state 136-1, indicating the user's readiness to use the implantable medical device 100 (e.g., to initiate deflation for voiding). A subsequent decrease in strain, below a second threshold or approaching a baseline level, may be interpreted as the user standing up or moving away. This change may be correlated with the user intent state 136-2, indicating the user's completion of use and the device's readiness to inflate.

To differentiate between actual user actions and minor seat movements or vibrations, in some examples, the system may incorporate a time-based analysis. Strain changes must be sustained for a minimum duration to be registered as a valid user intent state 136. The strain gage(s) 161 may be calibrated to detect strain changes corresponding to a user's weight with a certain sensitivity and resolution of microstrain.

In some examples, the sensor device 140 includes an impedance sensor 163 to detect the user's presence by measuring changes in electrical impedance. In some examples, a multi-electrode configuration is used to measure the user's body impedance at a certain frequency or range of frequences. The electrodes may be non-intrusively integrated into the surface of the toilet seat 270.

When the user sits on the toilet seat, their body completes an electrical circuit with the impedance sensor 163, resulting in a measurable change in impedance. The signal generator 134 may monitor the impedance. A decrease in impedance below a predefined threshold may be interpreted as the user contacting the seat. This impedance drop may be associated with user intent state 136-1, indicating the user's intention to use the device. An increase in impedance above a threshold, approaching the baseline level, is interpreted as the user breaking contact with the seat. This impedance rise may be associated with user intent state 136-2, indicating the user's departure and the device's readiness to transition. In some examples, the system may incorporate safety measures to limit the applied current to a maximum of microamps, ensuring user safety. Furthermore, to account for variations in skin conductivity and contact area, in some examples, the system analyzes the rate of change of impedance. A rapid impedance drop is given more weight than a slow change.

In some examples, the sensor device 140 includes a chemical sensor 165 that detects specific chemical markers present in urine. In some examples, the chemical sensor 165 is an electrochemical sensor. The chemical sensor 165 may measure changes in acidity (or basicity) (e.g., pH). In some examples, the chemical sensor 165 may measure the concentration of urea and creatinine. The chemical sensor 165 may be positioned within a recessed area in the toilet bowl to ensure contact with urine during voiding. The signal generator 134 may analyze the chemical sensor 165 output to identify specific patterns.

In some examples, a change (e.g., a significant change) in pH and the detection of urea and creatinine above predetermined concentration thresholds within a short timeframe (e.g., 5-10 seconds) may be interpreted as confirmation that voiding has occurred. In some examples, the chemical sensor 165 is a primary sensor in the sensor device 140. In some examples, the chemical sensor 165 is a secondary sensor in the sensor device 140. In some examples, while the primary user intent (to initiate voiding) is detected by other sensors, the chemical sensor 165 may provide a secondary confirmation of this intent, specifically the completion of the voiding action. In some examples, this confirmation can be used to more accurately identify the time the transition to user intent state 136-2.

In some examples, the sensor data from the chemical sensor 165 may be used in delaying the transition to the user intent state 136-2. In some examples, instead of immediately switching to the user intent state 136-2 when the user rises, the system may wait for the chemical sensor 165 to confirm voiding. This may prevent premature inflation if the user sits down but does not void. The chemical sensor 165 may be a response time of a certain time threshold (e.g., five seconds), meaning it provides a reading five seconds (or whichever threshold is used) after coming into contact with urine.

In some examples, to provide feedback to the user and/or facilitate interaction, the host device 168 may include an indicator 131. In some examples, the host device 168 may also include one or more user controls, allowing for confirmation and/or manual override of the automated functions. In some examples, the indicator 131 includes one or more visual indicators, such as light-emitting diodes (LEDs). Different colors, flashing patterns, or sequences of LEDs can convey various information, including confirmation of user intent state detection (e.g., a green LED indicates the detection of user intent state 136-1), activation or deactivation of the implantable medical device 100 (e.g., a blue LED pulsing during fluid transfer), or error conditions (e.g., a red LED flashing rapidly to indicate a sensor fault). In some examples, the indicator 131 may include a small display screen capable of showing text or simple graphics to provide more detailed information.

In some examples, the indicator 131 includes an audio output device, such as a speaker or buzzer. Different tones or sound patterns can be used to signal successful command reception, a low battery warning, or system errors. The volume and pitch of the audio feedback may be adjustable to suit user preferences. In some examples, the indicator 131 may include a haptic feedback device, such as a vibration motor. This can provide discreet feedback, such as a short vibration to confirm user intent detection or a longer vibration to signal the start or end of fluid transfer.

The host device 168 may also include user controls. In some examples, the host device 168 includes one or more confirmation controls to allow the user to acknowledge a detected user intent state 136 before the implantable medical device 100 is activated. This provides an additional layer of safety and prevents unintended activation. Examples of confirmation controls include a push-button, a touch-sensitive area, or a capacitive sensor that requires a sustained touch.

In some examples, the host device 168 may include override controls that allow the user or a caregiver to manually activate or deactivate the implantable medical device 100, bypassing the automated sensor-driven control. This may be beneficial in situations where the sensor data 132 is unreliable or in emergency scenarios. Override controls may include dedicated buttons for inflate and deflate, a key-operated switch to prevent unauthorized use, or a more complex interface with a display and menu options. In some examples, the host device 168 may include controls to adjust settings related to the implantable medical device 100, such as the pressure of the inflatable member 104, the sensitivity of the sensors 130, or the timing of fluid transfers.

The indicator 131 and any user controls are integrated into the host device 168 in a location that is easily visible and accessible to the user. For example, on a toilet seat, the indicator 131 may be placed on the side or the back of the seat, while controls may be located on a control panel at the front. On a urinal pad, the indicator 131 and controls may be integrated into a casing that surrounds the pad. In some examples, the pressure sensor 154 detects user presence, the indicator 131 (e.g., a green LED) flashes slowly to indicate detection, and the user presses a confirmation button to initiate deflation. In some examples, the system detects a low battery in the implantable medical device 100, and the indicator 131 (e.g., an audio buzzer) emits a series of beeps, prompting the user to recharge the device.

FIG. 2 illustrates an example of a host device 268. In the context of a urinary control device, the host device 268 is typically a bathroom fixture that the user interacts with during voiding. The term bathroom fixture or more generally host device could encompass components or structures associated with various types of toilet facilities found in different geographies, including variations of a hole in the ground or squat toilets. As long as there is a stable and predictable location (e.g., the surrounding platform, a dedicated mat, or a nearby wall/support) where sensors can be placed to detect relevant user interactions (like proximity, squatting posture, weight distribution, etc.) that indicate the intent to void, that structure or object could function as the host device for the sensor system. As shown in FIG. 2, the host device 268 may be a toilet cover 272, a toilet seat 270, or a toilet bowl 274. In some examples, the sensor device 140 of FIGS. 1A-1C may be integrated into or coupled to the toilet seat 270, the toilet cover 272, or the toilet bowl 274, or generally any portion of a toilet.

In some examples, a dedicated toilet seat 270 is installed in the patient’s home, replacing the standard one. In some examples, the sensor device can be coupled to (e.g., clipped onto) the toilet tank (or another part of the toilet). In some examples, the toilet seat 270 includes or is coupled to the sensor device which can detect the implantable medical device when the patient is seated on the toilet and ready for voiding. In some examples, the sensor device may be mounted inside the toilet bowl or inside a urinal. In some examples, the sensor device may be attached to the toilet at a location outside of the bowl (e.g., similar to a bidet control mechanism).

In some examples, the sensor device 140 is embedded within the seat's core material during manufacturing. In some examples, the sensor device 140 is attached to the top or bottom surface of the seat 270 using adhesive or mechanical fasteners. In some examples, a replacement toilet seat 270 is provided, specifically designed to house the sensor device 140 and provide enhanced user comfort, such as heating elements. In some examples, the sensor device 140 may be mounted on the inside surface of the bowl 274 using waterproof adhesive strips or suction cups. In some examples, the sensor device 140 may be integrated into a frame or housing that attaches to the rim of the bowl 274. In some examples, the sensor device 140 can be attached to the underside of the cover 272 or embedded within a pad or cushion affixed to the cover 272.

It should be understood that these are merely examples, and the host device 268 is not limited to toilet-related components. In some examples, the host device 268 may include a bathroom floor mat, a support frame or structure positioned around the toilet. In some examples, the sensor device 140 can be coupled to the host device 268 using various mechanisms, including mechanical coupling elements such as screws, bolts, clips and snap-fit connections, and/or adhesive bonding. The coupling mechanism may be permanent, designed to remain in place, or removable, allowing for detachment of the sensor device 140 for cleaning, maintenance, or replacement. The host device 268 is preferably constructed from materials that are resistant to moisture and easy to clean, such as plastics, ceramics, or coated metals.

FIG. 3 illustrates an example of a host device 368. The host device 268 may be a urinal pad 380. The host device 268 may include a urinal pad 380 (e.g., urinal cake), and, in some examples, a casing 382 that houses the urinal pad 380. In some examples, the sensor device 140 of FIGS. 1A-1C may be integrated into or coupled to the urinal pad.

In some examples, the host device 368 is a urinal pad 380, providing an alternative user interface for detecting user intent, particularly in public restroom settings. The urinal pad 380 is a porous block designed to be placed inside a urinal to reduce splashing and odors. It is typically made of a porous plastic material and shaped to fit the contours of a standard urinal. In some examples, the urinal pad 380 is housed within a casing 382. The casing 382 is a durable, non-reactive housing, such as one made of plastic or stainless steel, that protects the sensor device 140 from the harsh urinal environment. The casing 382 may have a mesh or perforated design to allow signals to pass through.

The sensor device 140 can be integrated into or coupled to the urinal pad 380. The sensor device 140 can be embedded within the material of the urinal pad 380 during the manufacturing process, requiring appropriate sealing and protection of the sensor. Alternatively, it can be attached to the surface of the urinal pad 380 or the interior of the casing 382 using adhesive, clips, or other fastening mechanisms.

Suitable sensor types for use with a urinal pad 380 include ultrasonic sensors, infrared sensors, capacitive sensors, and RFID sensors. For example, an ultrasonic sensor mounted at the top of the urinal pad 380 or casing 382, aimed upwards, can detect the presence of a user approaching or standing in front of the urinal. Capacitive sensors can be integrated into the surface of the urinal pad 380 to detect the user's proximity. User intent is inferred from the user's proximity to the urinal pad 380, as detected by the chosen sensor. The system can detect when a user approaches the urinal, stands in front of it, and potentially estimate the duration of urination based on the continued presence of the user. In some examples, the urinal pad 380 may include a light sensor, and, in some examples, a secondary confirmation that someone is standing in front of the urinal (e.g., the shadows would differ from normal ambient lighting).

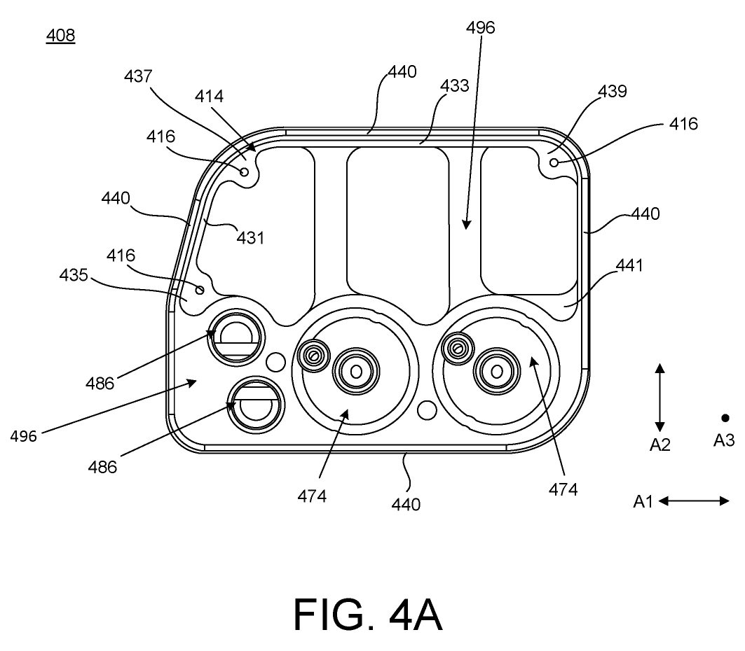

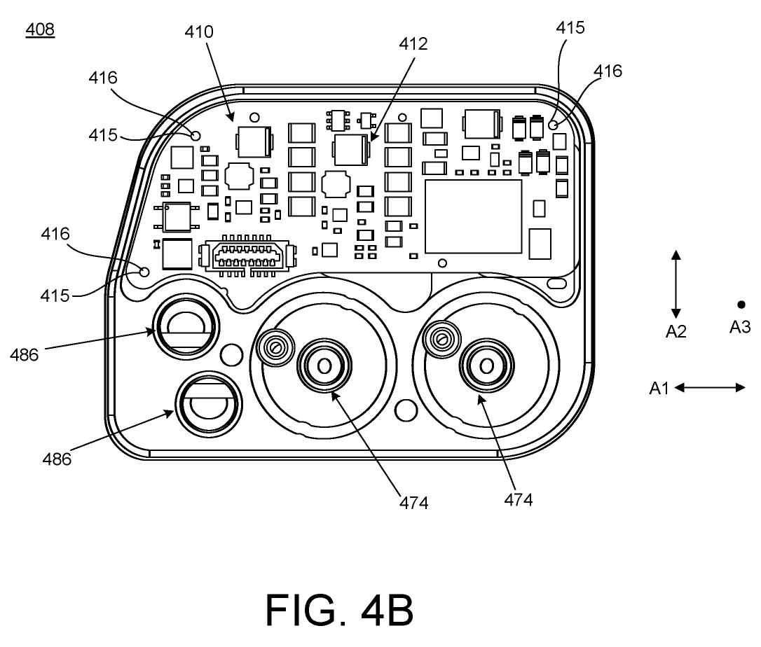

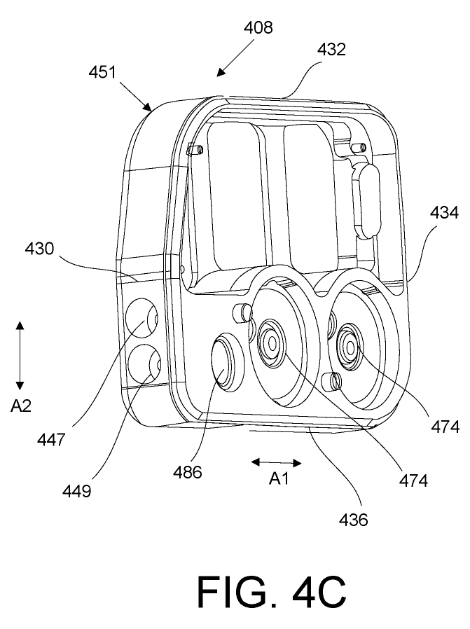

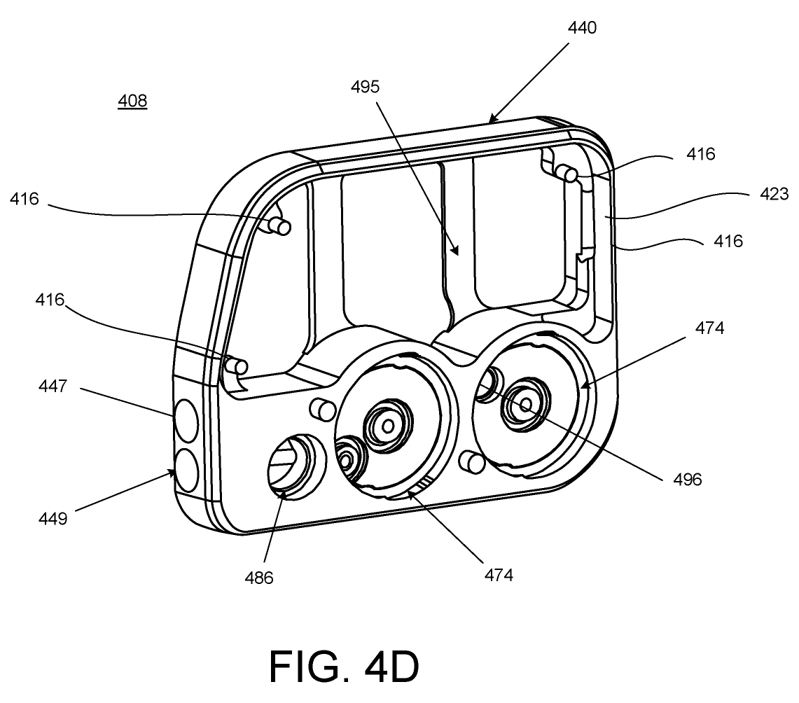

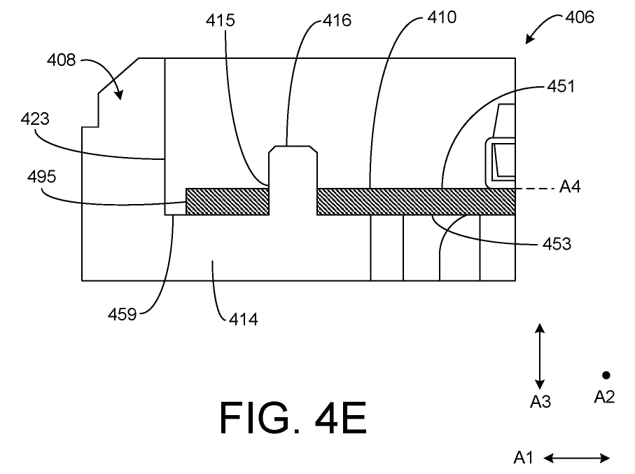

FIGS. 4A-4E illustrates a fluidic manifold of an electronic pump device 406 according to an aspect. The electronic pump device 406 may be an example of the electronic pump device 106 of FIGS. 1A-1C.

In some examples, the fluidic manifold 408 includes a conductive-based material (e.g., a metal-based material). In some examples, the fluidic manifold 408 is a titanium frame. The fluidic manifold 408 may attach a circuit substrate 410 with electronic components. The fluidic manifold 408 may attach one or more fluidic devices 474. A fluidic device 474 may be a pump. A fluidic device 474 may be a valve. The pump may be any of the pumps described in this disclosure. The valve may be any of the valves described in this disclosure. The fluidic manifold 408 includes a portion 496 configured to attach one or more pressure sensors 486 and one or more fluidic devices 474 (e.g., pump(s), valves). The fluidic manifold 408 includes a portion 496 configured to attach a circuit substrate 410 with electronic components 412. In some examples, the circuit substrate 410 is a printed circuit board (PCB). In some examples, the pressure sensors 486 include a first pressure sensor connected to a fluid reservoir to detect the pressure in the fluid reservoir, and a second pressure sensor connected to an inflatable member to detect the pressure in the inflatable member.

The fluidic manifold 408 includes a frame 440 with an inside edge 423. The fluidic manifold 408 includes a shelf 414 that extends from the inside edge 423. The circuit substrate 410 contacts the shelf 414 (e.g., a surface 459 of the shelf 414). The circuit substrate 410 may sit on top of the shelf 414 such that the circuit substrate 410 is positioned within the frame 440. The circuit substrate 410 includes a first surface 451 and a second surface 453 that is opposite to the first surface 451. As shown in FIG. 4E, the first surface 451 of the circuit substrate 410 is disposed in the plane A4. The thickness of the circuit substrate 410 is defined as the distance between the first surface 451 and the second surface 453 in the direction A3. The circuit substrate 410 includes an edge 495. The direction A3 is orthogonal to the plane A4. The direction A1 is perpendicular to the direction A3 and perpendicular to the direction A2. The direction A2 is perpendicular to the direction A1 and perpendicular to the direction A3. The second surface 453 of the circuit substrate 410 may contact at least a portion of the surface 459 of the shelf 414.

The fluidic manifold 408 includes one or more coupling members 416 that couples the circuit substrate 410 to the fluidic manifold 408 (e.g., to the shelf 414 of the fluidic manifold 408). In some examples, the coupling members 416 includes one or more male features (e.g., protrusions, posts, fasteners, rivets, etc.) and one or more female features (e.g., holes, slots, openings, etc.). The male feature may be defined on the fluidic manifold 408 (e.g., the shelf 414 of the fluidic manifold 408) and the female features may be circuit substrate 410. In some examples, the female feature may be defined on the fluidic manifold 408 (e.g., the shelf 414 of the fluidic manifold 408) and the male features may be defined on the circuit substrate 410.

In some examples, the coupling members 416 include protrusions (e.g., posts, cylindrical posts, grooved posts, extension members, etc.) that extend from the shelf 414 in the direction A3, and the protrusions extend through holes 415 on the circuit substrate 410. In some examples, the interaction between the protrusions and the holes 415 forms a press-fit coupling mechanism. Although some examples use a press-fit coupling mechanism, the circuit substrate 410, and the fluidic manifold 408 may be coupled to each other based on other types of coupling mechanisms.

The shelf 414 may include one or more shelf portions (e.g., also referred to as shoulder portions) that extend from the inside edge 423 of the frame 440 in the directions A1 and A2. The shelf 414 may have a thickness that extends in the direction A3. In some examples, the shelf portions extend from the inside edge 423 in the direction A2 and/or the direction A3 at multiple different lengths. The shelf portions may include a corner portion 435, a corner portion 437, a corner portion 439, and a corner portion 441. The corner portion 435, the corner portion 437, and the corner portion 441 may define, include, or contact a coupling member 416. The shelf portions may include a connecting portion 431 that extends between the corner portion 435 and the corner portion 437. The shelf portions may include a connecting portion 433 that extends between the corner portion 437 and the corner portion 439.

The frame 440 may define a peripheral wall formed by a wall portion 430, a wall portion 432, a wall portion 434, and a wall portion 436. In some examples, the outer surface of the wall portion 430, the wall portion 432, the wall portion 434, and the wall portion 436 form a portion of the outer surface of the electronic pump device. The fluidic manifold 408 includes a first fluid port 447 and a second fluid port 449. The first fluid port 447 and the second fluid port 449 are defined on the wall portion 430. A first tube member (e.g., tube member 103 of FIGS. 1A-1C) may be coupled to and extend from the first fluid port 447, and a second tube member (e.g., tube member 105 of FIGS. 1A-1C) may be coupled to and extend from the second fluid port 449. In some examples, the outer surface of the wall portion 430, the wall portion 432, the wall portion 434, and the wall portion 436 form a portion of the outer surface of the housing.

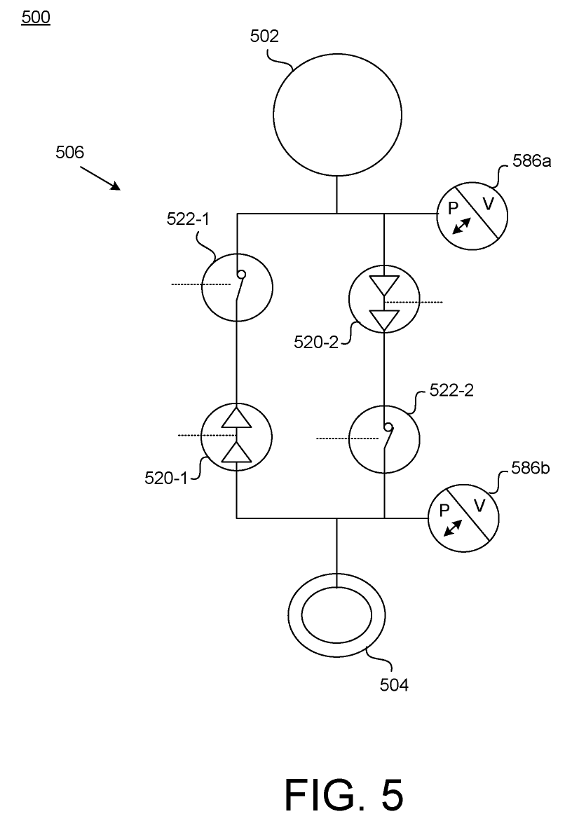

FIG. 5 illustrates an implantable medical device 500 according to an aspect. The implantable medical device 500 may be an example of the implantable medical device 100 of FIGS. 1A-1C and may include any of the details discussed herein. In some examples, the implantable medical device 500 is an artificial urinary sphincter device. The implantable medical device 500 includes a fluid reservoir 502, an inflatable member 504, and an electronic pump device 506 configured to transfer fluid between the fluid reservoir 502 and the inflatable member 504 to inflate or deflate the inflatable member 504. In some examples, the inflatable member 504 is an inflatable cuff.

The electronic pump device 506 includes a pressure sensor 586a connected to the fluid reservoir 502 and configured to monitor the pressure of the fluid reservoir 502, and a pressure sensor 586b connected to the inflatable member 504 and configured to monitor the pressure of the inflatable member 504.

The electronic pump device 506 includes a valve 522-1 and a pump 520-1. The valve 522-1 and the pump 520-1 are disposed in the same fluid passageway. The valve 522-1 and the pump 520-1 may be used to deflate the inflatable member 504. The electronic pump device 506 includes a pump 520-2 and a valve 522-2. The pump 520-2 and the valve 522-2 are disposed in the same fluid passageway. The valve 522-2 and the pump 520-2 may be used to inflate the inflatable member 504.

For example, in response to a deflate signal (e.g., when placed in a deflation mode), a controller may open the valve 522-1 and may activate the pump 520-1 to transfer fluid from the inflatable member 504 to the fluid reservoir 502. In the deflation mode, the valve 522-2 is closed, and the pump 520-2 is deactivated. In response to the pressure of the inflatable member 504 achieving a target deflate pressure, the controller may close the valve 522-1 and deactivate the pump 520-1. To maintain the target deflate pressure, the valve 522-1 and the valve 522-2 are closed. In response to an inflate signal (e.g., when placed in an inflation mode), a controller may open the valve 522-2 and may activate the pump 520-2. In the inflation mode, the valve 522-1 is closed, and the pump 520-1 is deactivated. In response to the pressure of the inflatable member 504 achieving a target inflate pressure, the controller may close the valve 522-2 and deactivate the pump 520-2. To maintain the target inflate pressure, the valve 522-1 and the valve 522-2 are closed. The controller may control the valve 522-1, the pump 520-1, the pump 520-2, and/or the valve 522-2.

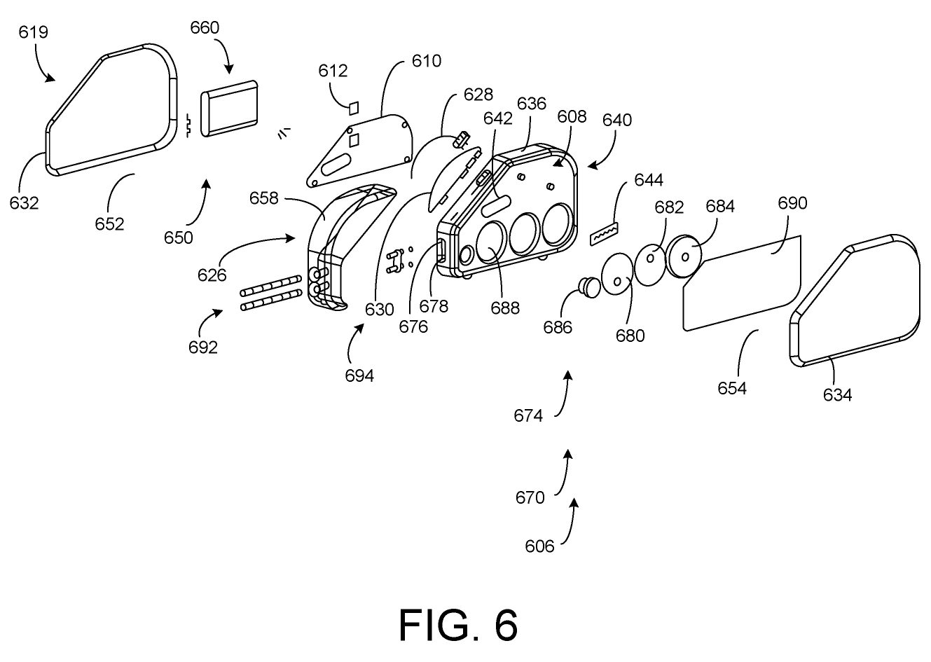

FIG. 6 illustrates an example of an exploded view of an electronic pump device 606 according to an aspect. The electronic pump device 606 may be an example of the electronic pump device 106 of FIGS. 1A-1C and may include any of the details discussed with reference to those figures. The housing 619 includes a first sidewall 632, a second sidewall 634, a peripheral wall 636, and a frame 640. The first sidewall 632, second sidewall 634, and the peripheral wall 636 are hermetically sealed together to form an internal compartment 650 within the housing 619.

The frame 640 is disposed within the internal compartment 650 to form a first partition 652 and a second partition 654 in such a manner that the first partition 652 is hermetically sealed from the second partition 654. The frame 640 can be integrally formed with the peripheral wall 636, the first sidewall 632, and/or the second sidewall 634. In some examples, the frame 640 is welded to the peripheral wall 636 or welded to the first sidewall 632, and/or the second sidewall 634. The first sidewall 632, the peripheral wall 636, and the frame 640 may form the first partition 652. The second sidewall 634, the peripheral wall 636, and the frame 640 may form the second partition 654, which is opposite the frame 640 from the first partition 652.

The electronic pump device 606 can include a header 626 attached to the housing 619 to form an internal region 658 between an inner surface of the header 626 and an outer surface of the housing 619 that includes power and communication interface structures such as a secondary coil 628 and the antenna 630 external to the hermetically sealed housing 619. The header 626 is configured from a dielectric or insulative material, such as a radome, to allow the transmission of power and communication signals between the antenna 630 and a handset programmer or charger, and between the secondary coil 628 and the charger. For example, the header 626 may include an over-molded polymer affixed to the housing 619 and including the secondary coil 628 and the antenna 630 within the internal region 658. The secondary coil 628 and antenna 630 are constructed from a biocompatible material. In some examples, the secondary coil 628 and antenna 630 can be formed as a coil from a stamped titanium core clad with gold or silver. In some examples, the secondary coil 628 and antenna 630 can be formed from a gold wire.

The electronic pump device 606 includes an energy storage system, such as a battery (e.g., a rechargeable power source) (e.g., a rechargeable battery), and electronic components 612 within the first partition 652. The electronic components 612 can be disposed on a circuit substrate 610, such as a plurality of circuit boards, within the first partition 652. The battery 660 can assume various forms appropriate to provide power for generating desired electrical signals and to store power provided from the electronic components 612. For example, the battery 660 can incorporate lithium-ion (Li+) chemistry, e.g., a lithium-ion battery to operate the electronic components 612. In some examples, the electronic components 612 can be implemented by various components including resistors, capacitors, transistors, and integrated circuits disposed on the circuit substrate 610. The secondary coil 628 and antenna 630 are electrically coupled to the electronic components 612 within the first partition 652, such as via a hermetic feedthrough component.

The electronic components 612 can include a recharge system, a communication system, and a controller. The recharge system includes hardware configured to interface with the secondary coil 628 to receive power signals, and to provide the power signals in a form suitable to recharge the battery 660 and can include circuitry to reduce the likelihood of overcharging the battery 660. The communication system includes hardware configured to interface with the antenna 630 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external controllers (e.g., external controller 101) (e.g., handset programmers).

The communication system can include an integrated circuit to implement an applied communication technology. In some examples, the communication system can be used to transmit communication signals to other devices, such as a charger or the handheld programmer (e.g., external controllers 101), and the communication system can be implemented to generate communication signals and provide the communication signals to the antenna 630 for transmission. In some examples, the communication system can be configured to receive and transmit radio frequency signals via the antenna 630. The controller can include a microcontroller to operate the recharge system and to receive and operate in response to communication signals or generate communication signals from the communication system.

The electronic pump device 606 also includes a fluidic circuit 670 within the second partition 654 and opposite the frame 640 from the battery 660 and electronic components 612. In some examples, the frame 640 can include an opening 642 that includes a hermetic interface 644, such as a feedthrough hermetically affixed to the frame 640. The electronic components 612 are operably coupled to the fluidic circuit 670 across the frame 640 via the hermetic interface 644. For example, the controller of the electronic components 612, powered by the battery 660, can cause the operation of the fluidic circuit 670 such as to control and monitor the fluidic circuit 670.

The fluidic circuit 670 includes a fluidic manifold 608 and fluidic devices 674 operably coupled to the fluidic manifold 608. In some examples, the fluidic manifold 608 is a structure integrated into the frame 640 such that the fluidic manifold 608 and the frame 640 together form the hermetic barrier between the first partition 652 and the second partition 654 of the internal compartment 650. For instance, the battery 660, the circuit substrate 610, or electronic components 612 can be coupled to a first major surface of the fluidic manifold 608 in the first partition 652, and the fluidic devices 674 are operably coupled to a second, and opposite major surface of the fluidic manifold 608 in the second partition 654.

The fluidic circuit 670 provides for the transfer of the fluid between the fluid reservoir (e.g., the fluid reservoir 102 of FIGS. 1A -1C) and the inflatable member (e.g., the inflatable member 104 of FIGS. 1A-1C). The fluidic manifold 608, which can be a hermetic manifold, segments and contains the fluid from the internal compartment 650 to reduce the chance of fluid exchange and directs the fluid from a first port 676 to a second port 678 via internal fluid passageways or channels.

The fluidic devices 674 include a plurality of fluid pumps, such as pumps 680, 682, a valve 684 mounted into the fluidic manifold 608 in fluidic communication with a manifold passageway to transfer fluid from the first port 676 to the second port 678. The pumps and the valve(s) are in fluid communication with a single fluid passageway between ports 676, 678. The fluidic devices 674 also include one or more pressure sensors 686 operably coupled to the fluidic manifold 608 and in fluidic communication with the passageway to detect the pressure of the fluid within the fluidic manifold 608.

The fluidic devices 674 are included in a planar configuration on the fluidic manifold 608 in which the pumps 680, 682, valve 684, and pressure sensor 686 are mounted into the fluidic manifold 608 on a plane for slim profile within the second partition 654. The fluidic manifold 608 can include chambers 688 formed into the second major surface in which the chambers are fluidically coupled to the single passageway within the fluidic manifold 608. The chambers are configured to receive the pumps 680, 682, and valve 684 and one or more pressure sensors 686. In some examples, the fluidic manifold 608 can receive a piezoelectric pump. The fluidic manifold 608 can receive a component cover 690 over the fluidic devices 674, which can be hermetically sealed to the second major surface.

In some examples, the electronic pump device 606 may include kink resistant tubing 692 that can extend through the header 626 and attached to the ports 676, 678 via components such as a barb 694 and O-rings. The kink resistant tubing 692 can be attached to the tube members 103, 105 of FIGS. 1A -1C to fluidically couple the electronic pump device 606 to the fluid reservoir and the inflatable member.

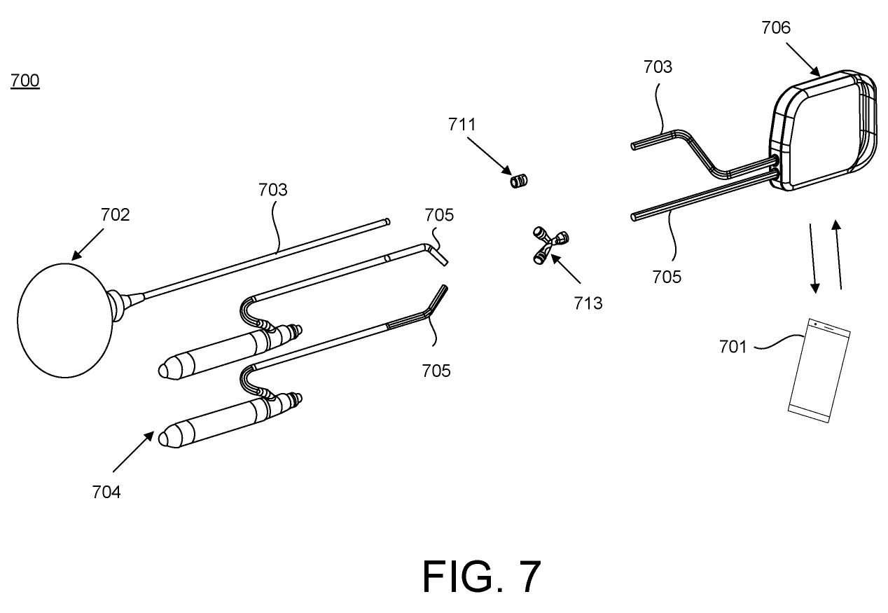

FIG. 7 illustrates a perspective of an inflatable penile prosthesis 700 according to an aspect. The inflatable penile prosthesis 700 may be an example of any of the medical devices discussed herein (e.g., including implantable medical device 100 of FIGS. 1A-1C), and, therefore, may include any of the details discussed with reference to the previous figures. The host device 168 (e.g., with the sensor device 140) or the sensor device 140 of FIGS. 1A-1C may be configured to communicate with the electronic pump device 706. In some examples, the sensor device 140 is a wearable device. In some examples, the sensor device 140 is integrated into an article of clothing or jewelry. In some examples, the sensor device 140 is a handheld device.

In some examples, the sensor device 140 is configured to detect user intent states related to inflation and deflation of the inflatable cylinders. To detect the user intent state (e.g., (intent to Inflate), the sensor device 140 may use proximity and gesture recognition. For example, capacitive sensors may be integrated into a handheld device that can detect specific hand movements or holding patterns. A prolonged hold of the device or a distinct gesture sequence may be interpreted as the user's intent to inflate the prosthesis. Alternatively, inertial measurement units (IMUs) within a wearable device can detect specific body movements or postural changes that correlate with the user's readiness for inflation. To detect the user intent state (e.g., intent to deflate), the sensor device 140 may detect the cessation of the gestures or movements associated with inflation. For instance, if the user moves the handheld device away from their body or returns to a resting posture, the sensor device 140 may interpret this as the intent to deflate. In some examples, using pressure sensors, a decrease in muscle tension or pressure could indicate the same intent. In some examples, voice commands can also be used. The user may speak commands such as “inflate” or “deflate,” which are captured by a microphone and processed by a speech recognition module to determine the appropriate user intent state.

The inflatable penile prosthesis 700 includes an inflatable member 704, a fluid reservoir 702, and an electronic pump device 706. The inflatable member 704 includes a pair of inflatable cylinders. The electronic pump device 706 may be an example of any of the pump devices discussed with reference to the previous figures and may include any of the details discussed herein. The electronic pump device 706 includes fluidic devices such as pumps, valves, and/or sensing devices positioned in fluid passageways. The electronic pump device 706 includes components such as, for example, one or more fluid control devices, one or more pressure sensors, and other such components. The electronic pump device 706 includes an electronic control system configured to provide for the transfer of fluid between a fluid reservoir 702 and an inflatable member 704 via the fluidic devices.

The electronic pump device 706 may include one or more integrated circuits. In some examples, the integrated circuits are included in a printed circuit board that is included in a housing of the electronic pump device 706. Fluidic devices and the electronic components of the electronic pump device 706 are included in a housing. In some examples, fluidic devices and electronic components in the housing define a manifold (e.g., an electronically controlled fluidic manifold) that provides for the electronic control of the flow of fluid between the fluid reservoir 702 and the inflatable member 704. In some examples, the electronic pump device 706 can communicate with an external controller 701, via respective communication modules. For example, an application stored in a memory and executed by a processor of the external controller 701 may allow the user and/or a physician to operate, view, monitor and alter operation of the inflatable penile prosthesis 700.

The inflatable penile prosthesis 700 includes one or more first tube members 703 that connect a first fluid port of the electronic pump device 706 with the fluid reservoir 702. One or more second tube members 705 connect a second fluid port of the electronic pump device 706 with the inflatable member 704 in the form of the inflatable cylinders. In some examples, the inflatable penile prosthesis 700 includes a connector 711 that is used to connect two tube members 703 together, and a connector 713 that is used to connect two tube members 705 together.

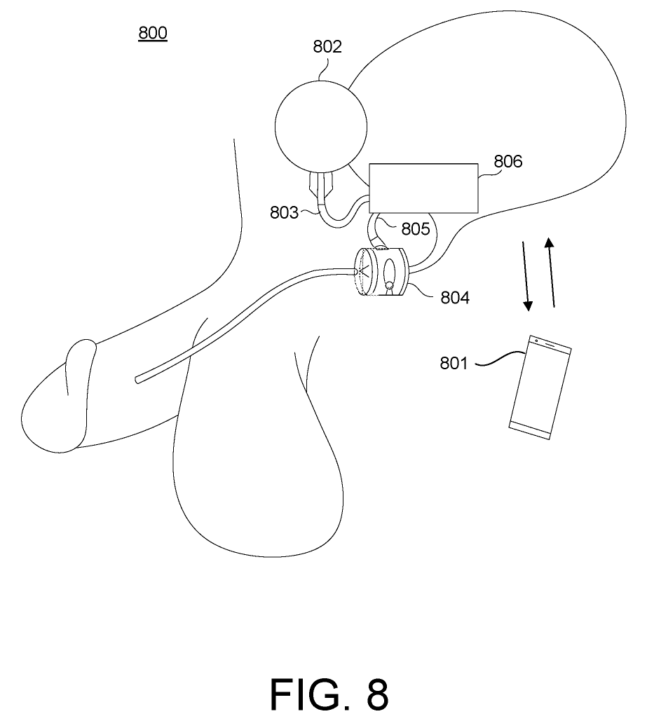

FIG. 8 illustrates a urinary control device 800 having an electronic pump device 806 according to an aspect. The urinary control device 800 may be an example of the implantable medical device 100 of FIGS. 1A-1C. In some examples, the urinary control device 800 is an artificial urinary sphincter device. The urinary control device 800 includes an electronic pump device 806, a fluid reservoir 802, and a cuff 804 (e.g., an inflatable cuff).

The fluid reservoir 802 may be a pressure-regulating inflation balloon or element. The fluid reservoir 802 is in operative fluid communication with the cuff 804 via one or more tube members 803, 805. The fluid reservoir 802 is constructed of polymer material that is capable of elastic deformation to reduce fluid volume within the fluid reservoir 802 and push fluid out of the fluid reservoir 802 and into the cuff 804. However, the material of the fluid reservoir 802 can be biased or include a shape memory construct adapted to generally maintain the fluid reservoir 802 in its expanded state with a relatively constant fluid volume and pressure. In some examples, this constant level of pressure exerted from the fluid reservoir 802 to the cuff 804 will keep the cuff 804 at a desired inflated state when open fluid communication is provided between the fluid reservoir 802 and the cuff 804. In some examples, the fluid reservoir 802 is implanted into the abdominal space.

A user may use an external controller 801 to control the urinary control device 800. In some examples, the user may use the external controller 801 to inflate or deflate the cuff 804. For example, in response to the user activating an inflation cycle using the external controller 801, the external controller 801 may transmit a wireless signal to the electronic pump device 806 to initiate the inflation cycle to transfer fluid from the fluid reservoir 802 to the cuff 804 (e.g., by opening an active valve where the pressure in the fluid reservoir 802 causes the fluid to move through the active valve to the cuff 804). In some examples, in response to the user activating a deflation cycle using the external controller 801, the external controller 801 may transmit a wireless signal to the pump device 806 to initiate the deflation cycle to transfer fluid from the cuff 804 to the fluid reservoir 802.

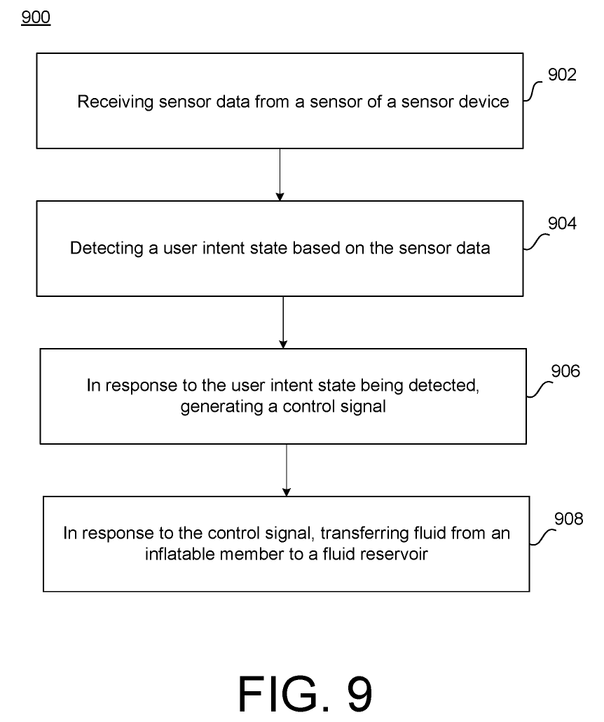

FIG. 9 illustrates a flowchart 900 depicting example operations of controlling a fluidic device of an electronic pump device of an implantable medical device according to an aspect. Although the flowchart 900 of FIG. 9 illustrates the operations in sequential order, it will be appreciated that this is merely an example, and that additional or alternative operations may be included. Further, operations of FIG. 9 and related operations may be executed in a different order than that shown, or in a parallel or overlapping fashion.

Operation 902 includes receiving sensor data from a sensor of a sensor device. In some examples, the sensor data 132 is received at the signal generator 134 of the sensor device 140. In some examples, the signal generator 134 is located in an electronic pump device 106 and the sensor data 132 is received at the electronic pump device 106. Operation 904 includes detecting a user intent state based on the sensor data. Operation 906 includes, in response to the user intent state being detected, generating a control signal. Operation 908 includes, in response to the control signal, transferring fluid from an inflatable member to a fluid reservoir.

Clause 1. An implantable medical device comprising: an inflatable member; a fluid reservoir; an electronic pump device configured to transfer fluid between the inflatable member and the fluid reservoir; and a sensor device configured to be integrated with or coupled to a host device, the sensor device configured to: receive sensor data; detect a user intent state based on the sensor data; and in response to the user intent state being detected, generate a control signal, the control signal configured to cause the electronic pump device to deflate the inflatable member.

Clause 2. The implantable medical device of clause 1, wherein the user intent state is a first user intent state and the control signal is a first control signal, wherein the sensor device is configured to: detect a second user intent state based on the sensor data; and in response to the second user intent state being detected, generate a second control signal, the second control signal configured to cause the electronic pump device to inflate the inflatable member.

Clause 3. The implantable medical device of clause 1 or 2, wherein the sensor device includes a transceiver configured to detect a wireless signal, the sensor device configured to detect the user intent state by detecting a presence of the wireless signal.

Clause 4. The implantable medical device of any one of clauses 1 to 3, wherein the sensor device includes a pressure sensor configured to generate a pressure reading about a pressure applied to the host device, the sensor device configured to detect the user intent state by detecting that the pressure reading is equal or greater than a threshold level.

Clause 5. The implantable medical device of any one of clauses 1 to 4, wherein the sensor device includes a radio-frequency identification (RFID) sensor, the sensor device configured to detect the user intent state by detecting RFID data from the RFID sensor.

Clause 6. The implantable medical device of any one of clauses 1 to 5, wherein the sensor device includes a capacitance sensor, the sensor device configured to detect the user intent state by detecting a change to a capacitance field.

Clause 7. The implantable medical device of any one of clauses 1 to 6, wherein the sensor device includes an inductive sensor, the sensor device configured to detect the user intent state by detecting a magnetic field.

Clause 8. The implantable medical device of any one of clauses 1 to 7, wherein the sensor device includes a light sensor, the sensor device configured to detect the user intent state by detecting a change of a luminance.

Clause 9. A method comprising: receiving sensor data from a sensor of a sensor device; detecting a user intent state based on the sensor data; in response to the user intent state being detected, generating a control signal; and in response to the control signal, transferring fluid from an inflatable member to a fluid reservoir.

Clause 10. The method of clause 9, wherein the user intent state is a first user intent state and the control signal is a first control signal, further comprising: detecting a second user intent state based on the sensor data; and in response to the second user intent state being detected, generating a second control signal; in response to the second control signal, transferring the fluid from the fluid reservoir to the inflatable member.

Clause 11. The method of clause 10, wherein the sensor data includes a wireless signal, wherein detecting the first user intent state includes detecting a presence of the wireless signal.

Clause 12. The method of clause 10 or 11, wherein the sensor data includes a wireless signal, wherein detecting the second user intent state includes detecting a lack of presence of the wireless signal.

Clause 13. The method of any one of clauses 10 to 12, wherein the sensor data includes a pressure reading about a pressure applied to a host device coupled to the sensor device, the sensor device configured to detect the first user intent state by detecting that the pressure reading is equal or greater than a threshold level.

Clause 14. The method of any one of clauses 10 to 13, wherein the sensor data includes a pressure reading about a pressure applied to a host device coupled to the sensor device, the sensor device configured to detect the second user intent state by detecting that the pressure reading is less than a threshold level.

Clause 15. The method of any one of clauses 9 to 14, wherein the sensor data includes radio-frequency identification (RFID) data, wherein detecting the user intent state based on the RFID data.

Clause 16. An implantable medical device comprising: an inflatable member; a fluid reservoir; an electronic pump device configured to transfer fluid between the inflatable member and the fluid reservoir; and a sensor device configured to be integrated with or coupled to a host device, the sensor device configured to: receive sensor data; detect a user intent state based on the sensor data; and in response to the user intent state being detected, generate a control signal, the control signal configured to cause the electronic pump device to deflate the inflatable member.

Clause 17. The implantable medical device of clause 16, wherein the user intent state is a first user intent state and the control signal is a first control signal, wherein the sensor device is configured to: detect a second user intent state based on the sensor data; and in response to the second user intent state being detected, generate a second control signal, the second control signal configured to cause the electronic pump device to inflate the inflatable member.

Clause 18. The implantable medical device of clause 16, wherein the sensor device includes a transceiver configured to detect a wireless signal, the sensor device configured to detect the user intent state by detecting a presence of the wireless signal.

Clause 19. The implantable medical device of clause 16, wherein the sensor device includes a pressure sensor configured to generate a pressure reading about a pressure applied to the host device, the sensor device configured to detect the user intent state by detecting that the pressure reading is equal or greater than a threshold level.

Clause 20. The implantable medical device of clause 16, wherein the sensor device includes a radio-frequency identification (RFID) sensor, the sensor device configured to detect the user intent state by detecting RFID data from the RFID sensor.

Clause 21. The implantable medical device of clause 16, wherein the sensor device includes a capacitance sensor, the sensor device configured to detect the user intent state by detecting a change to a capacitance field.

Clause 22. The implantable medical device of clause 16, wherein the sensor device includes an inductive sensor, the sensor device configured to detect the user intent state by detecting a magnetic field.

Clause 23. The implantable medical device of clause 16, wherein the sensor device includes a light sensor, the sensor device configured to detect the user intent state by detecting a change of a luminance.

Clause 24. A method comprising: receiving sensor data from a sensor of a sensor device; detecting a user intent state based on the sensor data; in response to the user intent state being detected, generating a control signal; and in response to the control signal, transferring fluid from an inflatable member to a fluid reservoir.

Clause 25. The method of clause 24, wherein the user intent state is a first user intent state and the control signal is a first control signal, further comprising: detecting a second user intent state based on the sensor data; and in response to the second user intent state being detected, generating a second control signal; in response to the second control signal, transferring the fluid from the fluid reservoir to the inflatable member.

Clause 26. The method of clause 25, wherein the sensor data includes a wireless signal, wherein detecting the first user intent state includes detecting a presence of the wireless signal.

Clause 27. The method of clause 25, wherein the sensor data includes a wireless signal, wherein detecting the second user intent state includes detecting a lack of presence of the wireless signal.

Clause 28. The method of clause 25, wherein the sensor data includes a pressure reading about a pressure applied to a host device coupled to the sensor device, the sensor device configured to detect the first user intent state by detecting that the pressure reading is equal or greater than a threshold level.

Clause 29. The method of clause 25, wherein the sensor data includes a pressure reading about a pressure applied to a host device coupled to the sensor device, the sensor device configured to detect the second user intent state by detecting that the pressure reading is less than a threshold level.

Clause 30. The method of clause 24, wherein the sensor data includes radio-frequency identification (RFID) data, wherein detecting the user intent state based on the RFID data.

Clause 31. An implantable medical device comprising: an electronic pump device configured to transfer fluid between an inflatable member and a fluid reservoir; and a sensor device configured to be integrated with or coupled to a host device, the sensor device configured to generate sensor data, the electronic pump device or the sensor device configured to: detect a user intent state based on the sensor data; in response to the user intent state being detected, generate a control signal; and in response to the control signal, initiate inflation or deflation of the inflatable member by activating one or more pumps of the electronic pump device.

Clause 32. The implantable medical device of clause 31, wherein the user intent state is a first user intent state and the control signal is a first control signal, wherein the sensor device or the electronic pump device is configured to: detect a second user intent state based on the sensor data; in response to the second user intent state being detected, generate a second control signal; and in response to the second control signal, initiate inflation or deflation of the inflatable member by activating one or more pumps of the electronic pump device.

Clause 33. The implantable medical device of clause 31, wherein the sensor device or the electronic pump device is configured to detect the user intent state by detecting a presence of a wireless signal.

Clause 34. The implantable medical device of clause 31, wherein the sensor device includes a pressure sensor configured to generate a pressure reading about a pressure applied to the host device, wherein the sensor device or the electronic pump device is configured to detect the user intent state by detecting that the pressure reading is equal or greater than a threshold level.

Clause 35. The implantable medical device of clause 31, wherein the sensor device includes a radio-frequency identification (RFID) sensor, the sensor device configured to detect the user intent state by detecting RFID data from the RFID sensor.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the disclosure. For example, the patient can be a person whose body is implanted with the medical device or the method disclosed for operating the medical device by the disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir;
an electronic pump device configured to transfer fluid between the inflatable member and the fluid reservoir; and
a sensor device configured to be integrated with or coupled to a host device, the sensor device configured to:
receive sensor data;
detect a user intent state based on the sensor data; and
in response to the user intent state being detected, generate a control signal, the control signal configured to cause the electronic pump device to deflate the inflatable member.

2. The implantable medical device of claim 1, wherein the user intent state is a first user intent state and the control signal is a first control signal, wherein the sensor device is configured to:
detect a second user intent state based on the sensor data; and
in response to the second user intent state being detected, generate a second control signal, the second control signal configured to cause the electronic pump device to inflate the inflatable member.

3. The implantable medical device of claim 1, wherein the sensor device includes a transceiver configured to detect a wireless signal, the sensor device configured to detect the user intent state by detecting a presence of the wireless signal.

4. The implantable medical device of claim 1, wherein the sensor device includes a pressure sensor configured to generate a pressure reading about a pressure applied to the host device, the sensor device configured to detect the user intent state by detecting that the pressure reading is equal or greater than a threshold level.

5. The implantable medical device of claim 1, wherein the sensor device includes a radio-frequency identification (RFID) sensor, the sensor device configured to detect the user intent state by detecting RFID data from the RFID sensor.

6. The implantable medical device of claim 1, wherein the sensor device includes a capacitance sensor, the sensor device configured to detect the user intent state by detecting a change to a capacitance field.

7. The implantable medical device of claim 1, wherein the sensor device includes an inductive sensor, the sensor device configured to detect the user intent state by detecting a magnetic field.

8. The implantable medical device of claim 1, wherein the sensor device includes a light sensor, the sensor device configured to detect the user intent state by detecting a change of a luminance.

9. A method comprising:
receiving sensor data from a sensor of a sensor device;
detecting a user intent state based on the sensor data;
in response to the user intent state being detected, generating a control signal; and
in response to the control signal, transferring fluid from an inflatable member to a fluid reservoir.

10. The method of claim 9, wherein the user intent state is a first user intent state and the control signal is a first control signal, further comprising:
detecting a second user intent state based on the sensor data; and
in response to the second user intent state being detected, generating a second control signal;
in response to the second control signal, transferring the fluid from the fluid reservoir to the inflatable member.

11. The method of claim 10, wherein the sensor data includes a wireless signal, wherein detecting the first user intent state includes detecting a presence of the wireless signal.

12. The method of claim 10, wherein the sensor data includes a wireless signal, wherein detecting the second user intent state includes detecting a lack of presence of the wireless signal.

13. The method of claim 10, wherein the sensor data includes a pressure reading about a pressure applied to a host device coupled to the sensor device, the sensor device configured to detect the first user intent state by detecting that the pressure reading is equal or greater than a threshold level.

14. The method of claim 10, wherein the sensor data includes a pressure reading about a pressure applied to a host device coupled to the sensor device, the sensor device configured to detect the second user intent state by detecting that the pressure reading is less than a threshold level.

15. The method of claim 9, wherein the sensor data includes radio-frequency identification (RFID) data, wherein detecting the user intent state based on the RFID data.

16. An implantable medical device comprising:
an electronic pump device configured to transfer fluid between an inflatable member and a fluid reservoir; and
a sensor device configured to be integrated with or coupled to a host device, the sensor device configured to generate sensor data,
the electronic pump device or the sensor device configured to:
detect a user intent state based on the sensor data;
in response to the user intent state being detected, generate a control signal; and
in response to the control signal, initiate inflation or deflation of the inflatable member by activating one or more pumps of the electronic pump device.

17. The implantable medical device of claim 16, wherein the user intent state is a first user intent state and the control signal is a first control signal, wherein the sensor device or the electronic pump device is configured to:
detect a second user intent state based on the sensor data;
in response to the second user intent state being detected, generate a second control signal; and
in response to the second control signal, initiate inflation or deflation of the inflatable member by activating one or more pumps of the electronic pump device.

18. The implantable medical device of claim 16, wherein the sensor device or the electronic pump device is configured to detect the user intent state by detecting a presence of a wireless signal.

19. The implantable medical device of claim 16, wherein the sensor device includes a pressure sensor configured to generate a pressure reading about a pressure applied to the host device, wherein the sensor device or the electronic pump device is configured to detect the user intent state by detecting that the pressure reading is equal or greater than a threshold level.

20. The implantable medical device of claim 16, wherein the sensor device includes a radio-frequency identification (RFID) sensor, the sensor device configured to detect the user intent state by detecting RFID data from the RFID sensor.
